(19) United States
(12) Patent Application Publication
(10) Pub. No.: US 2006/0194769 A1
(43) Pub. Date: Aug. 31, 2006

(54) Small molecules that reduce fungal growth

(75) Inventors: Douglas I. Johnson, Essex Junction, VT (US); Kurt A. Toenjes, Billings, MT (US)

Correspondence Address: WOLF GREENFIELD & SACKS, PC, FEDERAL RESERVE PLAZA, 600 ATLANTIC AVENUE, BOSTON, MA, 02210-2206, US

(73) Assignee: University of Vermont and State Agricultural College, Burlington, VT (US)

(21) Appl. No.: 11/340,418

(22) Filed: Jan. 25, 2006

Related U.S. Application Data
(60) Provisional application No. 60/646,967, filed on Jan. 25, 2005.

(51) Int. Cl. A61K 31/58; A61K 31/5513; A61K 31/551; A61K 31/55; A61K 31/517; A61K 31/47; A61K 31/445; A61K 31/44; A61K 31/366; A61K 31/353; A61K 31/405

(52) U.S. Cl. 514/114; 514/221; 514/457; 514/266.4; 514/620; 514/311; 514/176; 514/521; 514/218; 514/357; 514/562; 514/317; 514/455; 514/414; 514/560

(57) ABSTRACT

The present invention relates to methods for reducing the growth of a fungus with an anti-fungal small molecule. Methods for reducing fungal cell growth in a subject with an anti-fungal small molecule and related compositions are provided. Topical lotion formulations of an anti-fungal small molecule and a topical carrier are also provided.

### RELATED APPLICATIONS

This application claims priority to U.S. Provisional Application No. 60/646,967, filed Jan. 25, 2005, the entire contents of which are herein incorporated by reference.

### FIELD OF THE INVENTION

Methods for reducing the growth of a fungus with an anti-fungal small molecule are provided. Methods for treating fungal infection in a subject with an anti-fungal small molecule and related compositions are also provided. Compositions for reducing the growth of a fungus are provided. Topical lotion formulations of an anti-fungal small molecule and a topical carrier are also provided.

### BACKGROUND OF THE INVENTION

The invention is based on the discovery that anti-fungal small molecules can reduce the growth of a fungus. These anti-fungal small molecules can be used to treat a fungal infection, such as that caused by Candida albicans. C. albicans is the most common and arguably the most important causative agent of human fungal infections (Edmond, M. B. et al., 1999, Clin. Infect. Dis., 29:239-244). The yeast-to-hyphal morphological transition is essential for the virulence of C. albicans. It is a major opportunistic pathogen of immunocompromised hosts, including AIDS patients and those undergoing chemotherapy, tissue transplants or with central venous catheters. Studies indicate that up to 90% of AIDS patients suffer from oropharyngeal and esophageal candidiasis, in which C. albicans is the major causative agent (Schmidt-Westhausen, A. et al., 1991, J. Oral Pathol. Med., 20:467-472). Identification of novel anti-fungal targets is especially difficult as fungi are eukaryotic microbes that share many common cellular components with mammalian cells. Therefore, understanding processes unique to fungi, such as the yeast-to-hyphal transition, will undoubtedly lead to tremendous insight into virulence mechanisms and may ultimately lead to new anti-fungal therapeutic targets and drugs.

The need for new anti-fungal therapeutics is especially critical since there are serious side effects due to renal and liver dysfunction associated with the polyenes (i.e., amphotericin B, nystatin) that are usually used to treat C. albicans infections. In addition, a significant increase in resistance to the less toxic azole drugs (i.e., fluconoazole) has occurred within the patient population, especially HIV-positive patients.

### SUMMARY OF THE INVENTION

The invention provides in one aspect a method for reducing the growth of a fungus by contacting a cell with an anti-fungal small molecule in an amount effective to reduce the growth of a fungus. The invention also provides methods for treating a fungal infection in a subject by administering to a subject in need thereof an anti-fungal small molecule in an amount effective to reduce the growth of a fungus. Compositions for reducing the growth of a fungus are also provided. The compositions comprise an anti-fungal small molecule and an anti-fungal agent. Topical lotions are provided that comprise an anti-fungal small molecule and a topical carrier.

In an aspect of the invention a method for reducing fungal growth of a fungus by contacting a cell with an anti-fungal small molecule in an amount effective to reduce the growth of the fungal cell are provided.

In one aspect of the invention anti-fungal small molecules, analogs and salts thereof are provided. In one embodiment of the invention the anti-fungal small molecules have the following structures: 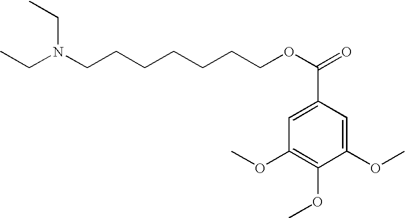
8-(N,N-Diethylamino)-octyl-3,4,5-trimethoxybenzoate, HCl (TMB-8) 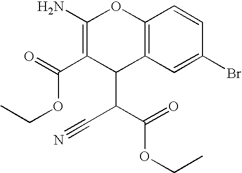
Ethyl[2-amino-6-bromo-4-(1-cyano-2-ethoxy-2-oxoethyl)]-4H-chromene-3-carboxylate (HA 14-1) 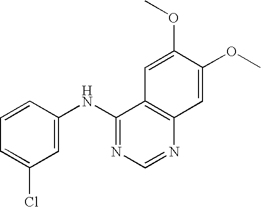
N-(3-Chlorophenyl)-6,7-dimethoxy-4-quinazolinamine (Tyrphostin AG1478) 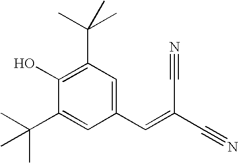
[[3,5-bis(1,1-Dimethylethyl)-4-hydroxyphenyl]methylene]propanedinitrile (Tyrphostin 9) 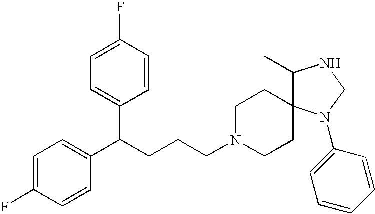
8-[4,4-bis(p-Fluorophenyl)butyl]-1-phenyl-1,3,8-triazino[4.5]decan-4-one (Fluspirilene) 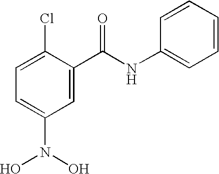
2-Chloro-5-nitro-N-phenylbenzamide(GW-9662) 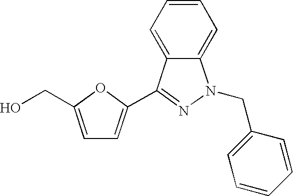
3-(5′-Hydroxymethyl-2′-furyl)-1-benzylindazole (YC-1). 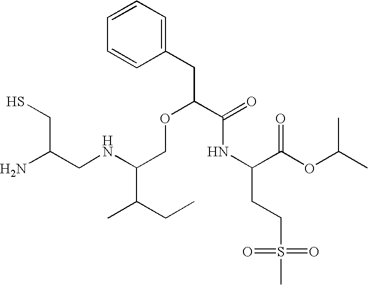
(2S)-2-[[(2S)-2-[(2S,3S)-2-[(2R)-2-Amino-3-mercaptopropyl]amino]-3-methylpentyl]oxy]-1-oxo-3-phenylpropyl]amino]-4-(methylsulfonyl)-butanoic Acid 1-Methylethyl Ester (L-744,832) 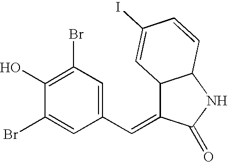
3-(3,5-Dibromo-4-hydroxybenzyliden)-5-iodo-1,2-dihydroindol-2-one (GW5074) 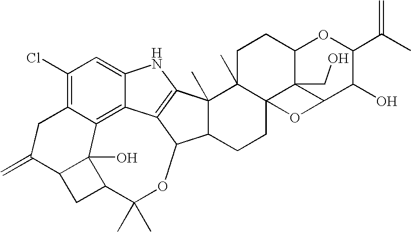
Penicillium palitans Penitrem A 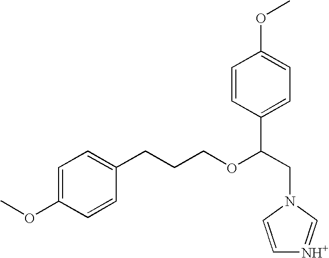
1-[β-[3-(4-Methoxyphenyl)propoxy]-4-methoxyphenethyl]-1H-imidazole, HCl (SKF-96365) 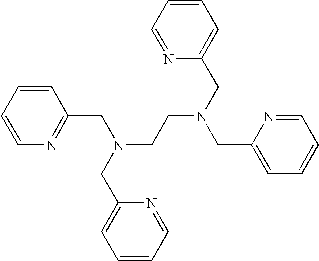
N,N,N′,N′-tetrakis-(2-pyridylmethyl)-ethylenediamine (TPEN) 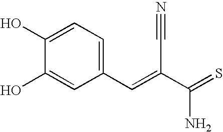
3,4-Dihydroxy-a-cyanothiocinnamamide, a-Cyano-3,4-dihydroxythiocinnamamide(Tyrphostin 47, AG213) 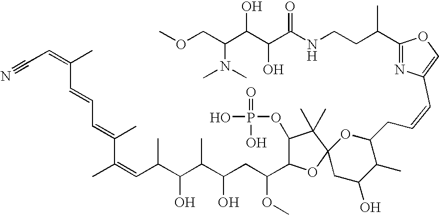
Discodermia calyx Calyculin A 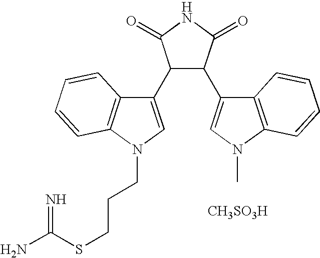
3-[1-[3-(Amidinothio)propyl-1H-indol-3-yl]-3-(1-methyl-1H-indol-3-yl)maleimide Bisindolylmaleimide IX, Methanesulfonate (Ro 31-8220) 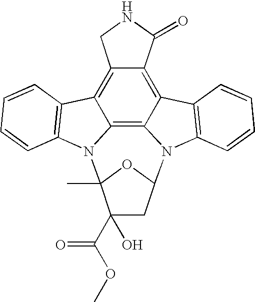
Cell permeable protein kinase inhibitor from Nocardiopsis (K252) 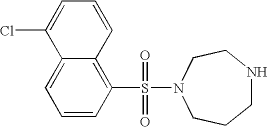
1-(5-Iodonapthalene-1-sulfonyl)homopiperazine (ML-7) 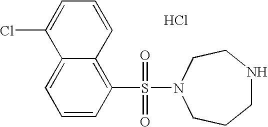
1-(5-Chloronapthalene-1-sulfonyl)homopiperazine, HCl (ML-9) 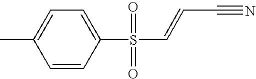
(E)3-[(4-Methylphenyl)sulfonyl]-2-propenenitrile (BAY 11-7082) 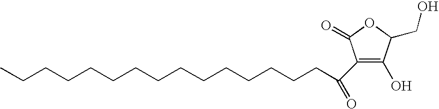
3-Hexadecanoyl-5-hydroxymethyl-tetronic Acid (RK-682) 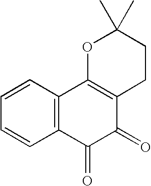
Tabebuia avellanedae β-lapachone 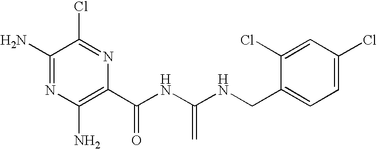
2,4-Dichlorobenzamil 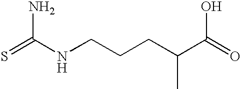
2-Thioureido-L-norvaline (Thiocitrulline) 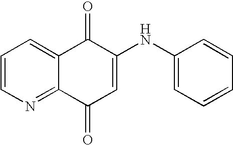
6-Anilino-5,8-quinolinequinone (LY-83583) 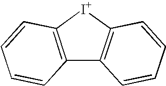
Diphenyleneiodonium 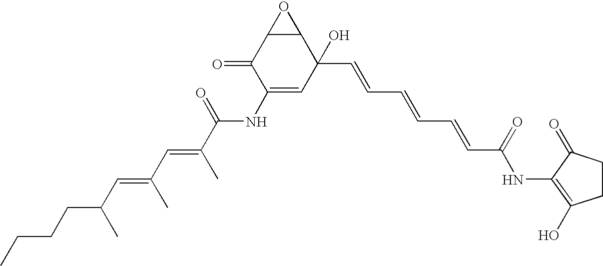
Manumycin A inhibitor of ras farnesyltransferase 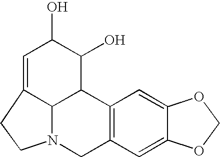
Lycorine 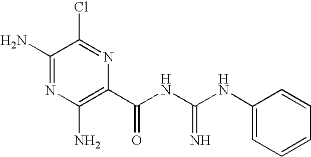
3,5-Diamino-6-chloro-N-[imino(phenylamino)methyl]pyrazinecarboxamide(Phenamil) 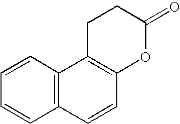
1,2-Dihydro-3H-naphtho[2,1-b]pyran-3-one (Splitomycin) 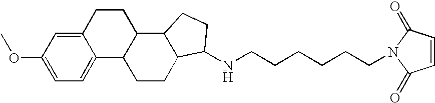
1-(6-(17β-3-methoxyestra-1,3,5(10)-trien-17-yl)amino)hexyl)-1H-pyrrole-2,5-dione (U73122) 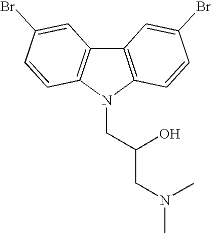
1-(3,6-Dibromocarbazol-9-yl)-3-dimethylaminopropan-2-ol (Wiskostatin) 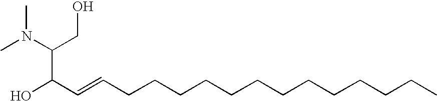
N,N-dimethylsphingosine 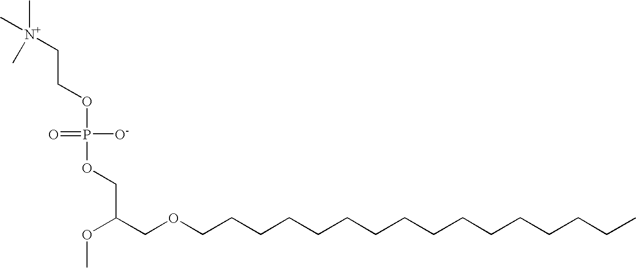
1-hexadecyl-2-methylglycero-3-phosphatidylcholine 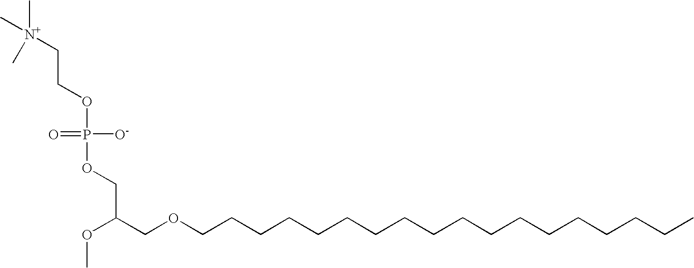
1-octadecyl-2-methylglycero-3 phosphatidylcholine 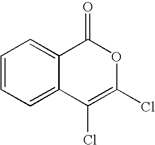
3,4-dichloroisocoumarin 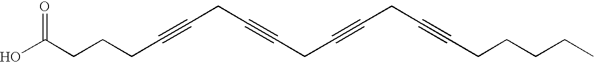
5,8,11,14-eicosatetrayonic acid 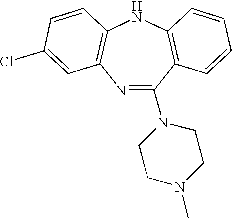
8-Chloro-11-(4-methyl-1-piperazinyl)-5H-dibenzo[b,e][1,4]-diazepine (Clozapine) 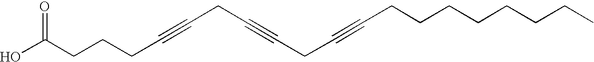
5,8,11-eicosatetrayonic acid 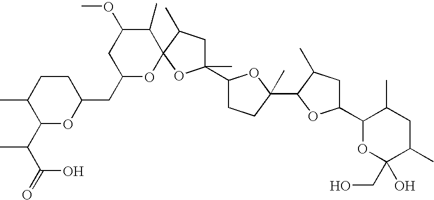
Streptomyces hygroscopicus (Nigericin) 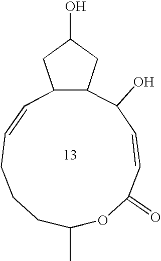
γ,4-Dihydroxy-2-(6-hydroxy-1-heptenyl)-4-cyclopentanecrotonic acidγ-lactone (Brefeldin A) 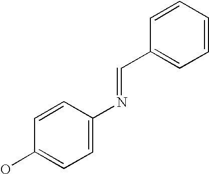
4-(Benzylidene-amino)-phenol 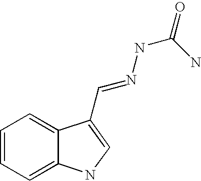 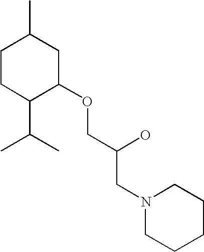
1-(2-Isopropyl-5-methyl-cyclohexyloxy)-3-piperidin-1-yl-propan-2-ol

In another aspect of the invention a method for treating a fungal infection in a subject by administering to a subject in need thereof an anti-fungal small molecule. In one embodiment the anti-fungal small molecule is one or more of 8-(N,N-Diethylamino)-octyl-3,4,5-trimethoxybenzoate HCl, Ethyl[2-amino-6-bromo-4-(1-cyano-2-ethoxy-2-oxoethyl)]-4H-chromene-3-carboxylate, N-(3-Chlorophenyl)-6,7-dimethoxy-4-quinazolinamine, [[3,5-bis(1,1-Dimethylethyl)-4-hydroxyphenyl]methylene]propanedinitrile, 8-Chloro-11-(4-methyl-1-piperazinyl)-5H-dibenzo[b,e][1,4]-diazepine, 8-[4,4-bis(p-Fluorophenyl)butyl]-1-phenyl-1,3,8-triazino[4.5]decan-4-one, 2-Chloro-5-nitro-N-phenylbenzamide, 3-(5′-Hydroxymethyl-2′-furyl)-1-benzylindazole, (2S)-2-[[(2S)-2-[(2S,3 S)-2-[(2R)-2-Amino-3-mercaptopropyl]amino]-3-methylpentyl]oxy]-1-oxo-3-phenylpropyl]amino]-4-(methylsulfonyl)-butanoic Acid 1-Methylethyl Ester, 3-(3,5-Dibromo-4-hydroxybenzyliden)-5-iodo-1,2-dihydroindol-2-one, 5,8,11,14-eicosatetrayonic acid, Penicillium palitans Penitrem A, 1-[β-[3-(4-Methoxyphenyl)propoxy]-4-methoxyphenethyl]-1H-imidazole HCl, N,N,N′,N′-tetrakis-(2-pyridylmethyl)-ethylenediamine, 3,4-Dihydroxy-a-cyanothiocinnamamide a-Cyano-3,4-dihydroxythiocinnamamide, Discodermia calyx Calyculin A, 3-[1-[3-(Amidinothio)propyl-1H-indol-3-yl]-3-(1-methyl-1H-indol-3-yl)maleimide Bisindolylmaleimide IX Methanesulfonate, Nocardiopsis K252A, 1-(5-Iodonapthalene-1-sulfonyl)homopiperazine, 1-(5-Chloronapthalene-1-sulfonyl)homopiperazine HCl, Streptomyces hygroscopicus Nigericin, γ,4-Dihydroxy-2-(6-hydroxy-1-heptenyl)-4-cyclopentanecrotonic acidγ-lactone, (E)3-[(4-Methylphenyl)sulfonyl]-2-propenenitrile, 3-Hexadecanoyl-5-hydroxymethyl-tetronic Acid, Tabebuia avellanedae Beta-lapachone, 2,4-Dichlorobenzamil, 2-Thioureido-L-norvaline, 6-Anilino-5,8-quinolinequinone, Diphenyleneiodonium Cl, Manumycin A, Lycorine, 3,5-Diamino-6-chloro-N-[imino(phenylamino)methyl]pyrazinecarboxamide, 1,2-Dihydro-3H-naphtho[2,1-b]pyran-3-one, 1-(6-(17β-3-methoxyestra-1,3,5(10)-trien-17-yl)amino)hexyl)-1H-pyrrole-2,5-dione, 1-(3,6-Dibromocarbazol-9-yl)-3-dimethylaminopropan-2-ol, N₁N-dimethylsphingosine, 1-hexadecyl-2-methylglycero-3-phosphatidylcholine, 1-octadecyl-2-methylglycero-3-phosphatidylcholine, 3,4-dichloroisocoumarin, 5,8,11-eicosatriyonic acid, 4-(Benzylidene-amino)-phenol, 1-(2-isopropyl-5-methyl-cyclohexyloxy)-3-piperidin-1-yl-propan-2-ol, or 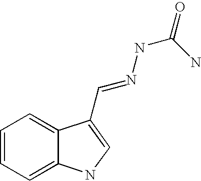
and analogs and salts thereof.

In an embodiment of the invention the anti-fungal small molecule is 4-(benzylidene-amino)-phenol and 2-Chloro-5-nitro-N-phenylbenzamide analogs or salts thereof. In another embodiment of the invention the anti-fungal small molecule is 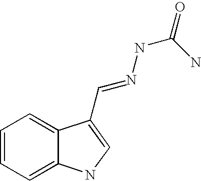
and 8-Chloro-11-(4-methyl-1-piperazinyl)-5H-dibenzo[b,e][1,4]-diazepine analogs or salts thereof. In a further embodiment of the invention the anti-fungal small molecule is 1-(2-isopropyl-5-methyl-cyclohexyloxy)-3-piperidin-1-yl-propan-2-ol and 8-Chloro-11-(4-methyl-1-piperazinyl)-5H-dibenzo[b,e][1,4]-diazepine analogs or salts thereof. In another embodiment of the invention the anti-fungal small molecule is 4-(benzylidene-amino)-phenol and ethyl[2-amino-6-bromo-4-(1-cyano-2-ethoxy-2-oxoethyl)]-4H-chromene-3-carboxylate.

In an embodiment of the invention the fungal infection is one or more of Candida albicans, Pneumocystis carinii, Saccharomyces cerevisiae, Aspergillus nidulans, Kluyveromyces lactis, Schizosaccharomyces pombe, Streptomyces lasaliensis, Streptomyces hygroscopicus, Candida tropicalis, Candida dubliniensis, Candida parapsilosis, Candida kefyr, Candida guilliermondii, Candida inconspicua, Candida famata, Candida glabrata, Candida krusei, Candida lusitaniae, Cryptococcus neoformans, Coccidioides immitis, or Hispolasma capsulatum. In a second embodiment the fungal infection is a pathogenic yeast. In another embodiment the fungal infection is Candida albicans.

In one embodiment of the invention the subject is a human. In another embodiment the subject is immunocompromised. In another embodiment the subject has had chemotherapy. In a further embodiment the subject has AIDS. In still further embodiments the subject has a central venous catheter.

In an embodiment of the invention the anti-fungal molecule is administered via injection, topical route, oral route, nasal route, aerosol, or enema route. In one embodiment the anti-fungal small molecule is administered via an oral route. In a second embodiment the anti-fungal small molecule is administered via a topical route.

In an aspect of the invention a composition comprising an anti-fungal small molecule and an anti-fungal agent is provided, which may optionally be a topical lotion. A topical lotion comprising an anti-fungal small molecule and a topical carrier is also provided according to other aspects of the invention. In some embodiments the anti-fungal small molecule is one or more of the anti-fungal small molecules listed above and/or described herein.

In an embodiment of the invention the anti-fungal agent is an anti-Candida albicans agent. In a second embodiment the anti-Candida albicans agent is one or more of Acrisorcin; Ambruticin; Amphotericin B; Azaconazole; Azaserine; Basifungin; Bifonazole; Biphenamine Hydrochloride; Bispyrithione Magsulfex; Butoconazole Nitrate; Calcium Undecylenate; Candicidin; Carbol-Fuchsin; Chlordantoin; Ciclopirox; Ciclopirox Olamine; Cilofungin; Cisconazole; Clotrimazole; Cuprimyxin; Denofungin; Dipyrithione; Doconazole; Econazole; Econazole Nitrate; Enilconazole; Ethonam Nitrate; Fenticonazole Nitrate; Filipin; Fluconazole; Flucytosine; Fungimycin; Griseofulvin; Hamycin; Isoconazole; Itraconazole; Kalafungin; Ketoconazole; Lomofungin; Lydimycin; Mepartricin; Miconazole; Miconazole Nitrate; Monensin; Monensin Sodium; Naftifine Hydrochloride; Neomycin Undecylenate; Nifuratel; Nifurmerone; Nitralamine Hydrochloride; Nystatin; Octanoic Acid; Orconazole Nitrate; Oxiconazole Nitrate; Oxifungin Hydrochloride; Parconazole Hydrochloride; Partricin Potassium Iodide; Proclonol; Pyrithione Zinc; Pyrrolnitrin; Rapamycin; Rutamycin; Sanguinarium Chloride; Saperconazole; Scopafungin; Selenium Sulfide; Sinefungin; Sulconazole Nitrate; Tamoxifen; Terbinafine; Terconazole; Thiram; Ticlatone; Tioconazole; Tolciclate; Tolindate; Tolnaftate; Triacetin; Triafungin; Tunicamycin; Undecylenic Acid; Viridofulvin; Zinc Undecylenate; or Zinoconazole Hydrochloride.

In one embodiment of the invention the topical lotion is formulated as a cream, an ointment, drops, a gel, a controlled-release patch, a spray, a pessary, or a foam.

In an embodiment of the invention the topical carrier is one or more of mineral oil, sorbitan monostearate, polysorbate 60, cetyl esters wax, cetearyl alcohol, 2-octyldodecanol, benzyl alcohol, liquid petroleum, white petroleum, propylene glycol, polyoxyethylene polyoxypropylene compound, emulsifying wax or water.

### DETAILED DESCRIPTION OF THE INVENTION

Methods for reducing the growth of a fungus using anti-fungal small molecules have been discovered. Applicants have discovered that specific classes of molecules are effective in reducing the growth and for inhibiting the yeast-to-hyphal transition of fungi. These molecules are thus, useful for a variety of in vitro and in vivo uses, such as those described herein.

In an aspect of the invention a fungal cell is contacted with an anti-fungal small molecule in an amount effective to reduce the fungal cell growth. It is intended that the fungal cell is contacted either in vitro or in situ, whereby in situ includes contacting a fungal cell in vivo or contacting a fungal cell on the surface of the skin. One of ordinary skill in the art would understand “contacting” to encompass putting a fungal cell into contact with an anti-fungal small molecule for example in a tissue culture plate whereby the fungal cell is placed into a media environment and an anti-fungal small molecule is added to the media. Further “contacting” would be understood by one of ordinary skill in the art to mean adding an anti-fungal small molecule to a fungal cell or population of fungal cells on the surface of the skin or parenterally or locally applying an anti-fungal agent to a subject such that the fungus in the subject is exposed to the anti-fungal agent.

A fungal cell is intended to encompass any cell originating from a fungal species or fungus. As used herein a fungus is also intended to include moulds, yeast and pathogenic yeast. A fungus includes but is not limited to Candida for example Candida albicans, Candida tropicalis, Candida dubliniensis, Candida parapsilosis, Candida kefyr, Candida guilliermondii, Candida inconspicua, Candida famata, Candida glabrata, Candida krusei, and Candida lusitaniae, Pneumocystis for example Pneumocystis carinii, Saccharomyces for example Saccharomyces cerevisiae, Aspergillus for example Aspergillus nidulans, Kluyveromyces for example Kluyveromyces lactis, Schizosaccharomyces for example Schizosaccharomyces pombe, and Streptomyces for example Streptomyces lasaliensis and Streptomyces hygroscopicus, Cryptococcus neoformans, Coccidioides immitis, and Hispolasma capsulatum. In some embodiments the fungus is a pathogenic yeast, such as Candida albicans.

In some instances the compounds described herein are useful also for treating a fungal infection in a subject. As used herein treating or treat is intended to include preventing, ameliorating, curing, reducing fungal growth or reducing symptoms, or preventing any increase in fungal growth or symptoms.

As used herein “reducing fungal growth” is intended to encompass an interference in fungal cell growth or processing which can be determined by a reduction in cell number, a reduction in cell division or a reduction in the yeast-to-hyphal transition phase.

Methods for detecting a reduction in fungal growth are known to those of skill in the art and include high throughput assays. A novel example of such a method is described in U.S. application Ser. No. 60/445,314 and corresponding PCT application serial no. PCT[US04/03208 designating the US, both of which are incorporated herein by reference. Generally fungal cells are grown in microtitre plates and incubated with a molecule of the invention to determine reduction of fungal cell growth. For example, C. albicans cells are grown in YNB media that inhibits hyphal growth and then transferred to 384-well optical plates containing Spider media to induce the budded-to-hyphal transition and hyphal elongation. The yeast-to-hyphal morphological transition is essential for the virulence of C. albicans. An anti-fungal small molecule is added, incubated at 37° C. for 4 hours and inhibition or reduction of fungal growth determined. One of skill in the art would be able to detect a reduction in growth by routine methods such as microscopy. YNB media is well known in the art and contains yeast nitrogen base (DIFCO Labs.), glucose (US Biological) and d-H₂O. Spider media is well known in the art and contains nutrient broth (DIFCO Labs.), mannitol (Sigma-Aldrich), K₂HPO₄ (Sigma-Aldrich) and d-H₂O.

Other methods for determining a reduction in fungal growth include methods for determining the number of fungal cells using cell staining techniques such as trypan blue and counting the cells using a microscope. Methods such as PCR and RT-PCR are contemplated for determining a reduction of RNA or DNA as a measure of reduced fungal growth. Other methods include observation of a visible reduction of the fungal infection as a result of reduced fungal growth. These methods are well known to those of ordinary skill in the art and require routine procedures.

A “subject” as used herein is any animal in need of treatment, including humans, primates and other mammals such as equines, cattle, swine, sheep, goats, primates, mice, rats, and pets in general including dogs, cats, guinea pigs, ferrets, and rabbits.

As used herein subject in need thereof is a subject having a fungal infection, or a subject at risk of developing a fungal infection. The subject may have been diagnosed as having such a fungal infection as described herein or using standard medical techniques known to those of skill in the art. Alternatively a subject may exhibit one or more symptoms of fungal infection.

A subject at risk of developing a fungal infection is a subject who has been exposed to a fungus, or is susceptible to exposure to a fungus. For instance a subject that is susceptible to exposure to a fungus includes those subjects who work with fungal material or in areas of high fungal content, subjects who travel to areas with high fungal infectivity rates or are otherwise likely to be exposed to a fungal infection as well as those subjects having particular susceptibility to fungal infection resulting from medical conditions or therapies. Examples of subjects having particular susceptibility to fungal infections arising from medical conditions or therapies include but are not limited to an immunocompromised subject, a subject having received chemotherapy, a subject having cancer, a subject having AIDS, a subject who is HIV positive, a subject who is at risk of being HIV positive, a subject having received a transplant, or a subject having a central venous catheter.

An immunocompromised subject is a subject that is incapable of inducing a normal effective immune response or a subject that has not yet developed an immune system (e.g. preterm neonate). An immunocompromised subject, for example, is a subject undergoing or undergone chemotherapy, a subject having AIDS, a subject receiving or having received a transplant or other surgical procedure etc.

A subject having received chemotherapy is a subject that has undergone some form of chemotherapeutic procedure. Chemotherapeutic procedure encompasses conventional methods known to those of skill in the art. Examples of chemotherapeutic methods include but are not limited to alkylating agents, for example, nitrogen mustards, ethyleneimine compounds and alkyl sulphonates; antimetabolites, for example, folic acid, purine or pyrimidine antagonists, mitotic inhibitors, for example, vinca alkaloids and derivatives of podophyllotoxin; cytotoxic antibiotics; compounds that damage or interfere with DNA expression; and growth factor receptor antagonists; antibodies and other biological molecules known to those of ordinary skill in the art.

A subject having cancer is a subject that has detectable cancerous cells. The cancer may be a malignant or non-malignant cancer. Cancers or tumors include but are not limited to biliary tract cancer; brain cancer including glioblastomas and medulloblastomas; bladder cancer; breast cancer; cervical cancer; choriocarcinoma; colon cancer including colorectal carcinomas; endometrial cancer; esophageal cancer; gastric cancer; head and neck cancer; hematological neoplasms including acute lymphocytic and myelogenous leukemia; AIDS-associated leukemias and adult T-cell leukemia lymphoma; intraepithelial neoplasms including Bowen's disease and Paget's disease; lymphomas including Hodgkin's disease and lymphocytic lymphomas; liver cancer; lung cancer (e.g. small cell and non-small cell); melanoma; neuroblastomas; multiple myeloma; oral cancer including squamous cell carcinoma; osteosarcomas; ovarian cancer including those arising from epithelial cells, stromal cells, germ cells and mesenchymal cells; pancreas cancer; prostate cancer; rectal cancer; sarcomas including leiomyosarcoma, rhabdomyosarcoma, liposarcoma, fibrosarcoma, synovial sarcoma and osteosarcoma; skin cancer including melanomas, Kaposi's sarcoma, basocellular cancer, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; transitional cancer and renal cancer including adenocarcinoma and Wilms tumor, as well as other carcinomas and sarcomas.

A subject who is HIV positive encompasses a subject who is a carrier of any of the HIV family of retroviruses or a subject who is diagnosed of active AIDS, as well as a subject having AIDS-related conditions. A carrier of HIV may be identified by any methods known in the art. For example, a subject can be identified as an HIV carrier on the basis that the subject is anti-HIV antibody positive, or is HIV-positive, or has symptoms of AIDS. HIV infection generally encompasses infection of a host, particularly a human host, by the human immunodeficiency virus (HIV) family of retroviruses including, but not limited to, HIV I, HIV II, HIV III (also known as HTLV-II, LAV-1, LAV-2), and the like. “HIV” can be used herein to refer to any strains, forms, subtypes and variations in the HIV family. A subject having HIV is a subject who is at any one of the several stages of HIV infection progression, which, for example, include acute primary infection syndrome (which can be asymptomatic or associated with an influenza-like illness with fevers, malaise, diarrhea and neurologic symptoms such as headache), asymptomatic infection (which is the long latent period with a gradual decline in the number of circulating CD4+ T cells), and AIDS (which is defined by more serious AIDS-defining illnesses and/or a decline in the circulating CD4 cell count to below a level that is compatible with effective immune function). In addition, it is intended to encompass subjects suspected of being infected with HIV after suspected past exposure to HIV by e.g., contact with HIV-contaminated blood, blood transfusion, exchange of body fluids, “unsafe” sex with an infected subject, accidental needle stick, receiving a tattoo or acupuncture with contaminated instruments, or transmission of the virus from a mother to a baby during pregnancy, delivery or shortly thereafter. Subjects who are HIV positive also encompass subjects who have not been diagnosed as having HIV infection but are believed to be at high risk of infection by HIV.

A subject having acquired immunodeficiency syndrome (AIDS) is a subject who exhibits more serious AIDS-defining illnesses and/or a decline in the circulating CD4 cell count to below a level that is compatible with effective immune function. A subject having AIDS also encompasses a subject having AIDS-related conditions, which means disorders and diseases incidental to or associated with AIDS or HIV infection such as AIDS-related complex (ARC), progressive generalized lymphadenopathy (PGL), anti-HIV antibody positive conditions, and HIV-positive conditions, AIDS-related neurological conditions (such as dementia or tropical paraparesis), Kaposi's sarcoma, thrombocytopenia purpurea and associated opportunistic infections such as Pneumocystis carinii pneumonia, Mycobacterial tuberculosis, esophageal candidiasis, toxoplasmosis of the brain, CMV retinitis, HIV-related encephalopathy, HIV-related wasting syndrome, etc.

A subject having received a transplant is a subject having received either a tissue or organ transplant during a surgical procedure. Transplants include but are not limited to organ, tissue, stem cell, bone marrow, and encompass conventional methods known to those of skill in the art. A subject having received a tissue transplant is especially susceptible to fungal infections from Candida species such as Candida albicans.

A subject having a central venous catheter is a subject having received a central venous catheter implant during a surgical procedure. A central venous catheter implant encompasses the use of conventional methods known to those of skill in the art. A subject having received a central venous catheter is especially susceptible to fungal infections from Candida species such as Candida albicans.

In an aspect of the invention an anti-fungal small molecule, analog or salt thereof has the following structure:

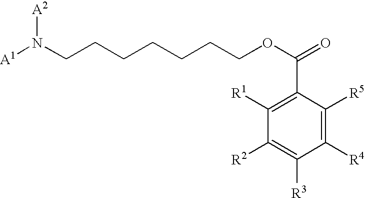

In an embodiment each of A¹ and A² independently is one of —H or an alkyl; and each of R¹, R², R³, R⁴, and R⁵ independently is one of —H, a halogen, an alkyl, —OH, or an alkoxy. In a second embodiment each of A¹ and A² independently is an alkyl. In another embodiment each of A¹ and A² is ethyl. In one embodiment at least one of R¹, R², R³, R⁴, or R⁵ is an alkoxy. In a second embodiment at least two of R¹, R², R³, R⁴, or R⁵ independently is an alkoxy. In another embodiment at least three of R¹, R², R³, R⁴, or R⁵ independently is an alkoxy. In a further embodiment at least one of R¹, R², R³, R⁴, or R⁵ is methoxy. In yet another embodiment each of R², R³, and R⁴ is methoxy. In further embodiments each of R¹ and R⁵ is —H. In a preferred embodiment the anti-fungal small molecule has the following structure:

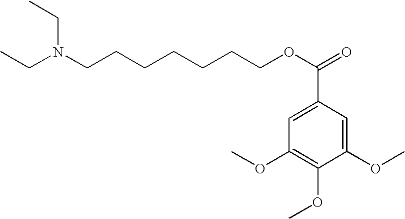

In an aspect of the invention an anti-fungal small molecule, analog or salt thereof has the following structure:

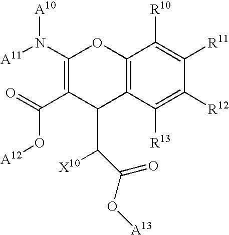

In an embodiment each of A¹⁰, A¹¹, A¹², and A¹³ independently is one of —H or an alkyl; X¹⁰ is —CN or a halogen; and each of R¹⁰, R¹¹, R¹², and R¹³ independently is one of —H, a halogen, an alkyl, —OH, or an alkoxy. In a second embodiment at least one of R¹⁰, R¹¹, R¹², or R¹³ is a halogen. In another embodiment at least one of R¹⁰, R¹¹, R¹², or R¹³ is —Br. In a further embodiment R¹² is —Br. In yet another embodiment each of R¹⁰, R¹¹, and R¹³ is —H. In still another embodiment X¹⁰ is —CN. In an embodiment each of A¹⁰ and A¹¹ is —H. In a second embodiment A¹² is an alkyl. In another embodiment A¹² is ethyl. In a further embodiment A¹³ is an alkyl. In yet another embodiment A¹³ is ethyl. In a preferred embodiment the anti-fungal small molecule has the following structure:

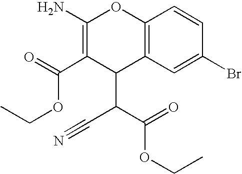

In an aspect of the invention an anti-fungal small molecule, analog or salt thereof has the following structure:

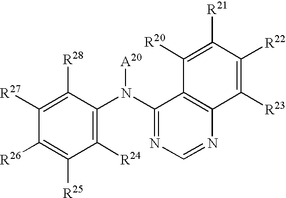

In an embodiment A²⁰ is one of —H or an alkyl; and each of R²⁰, R²¹, R²², R²³, R²⁴, R²⁵, R²⁶, R²⁷, and R²⁸ independently is one of —H, a halogen, an alkyl, —OH, or an alkoxy. In a second embodiment A²⁰ is —H. In another embodiment at least one of R²⁰, R²¹, R²², or R²³ is an alkoxy. In a further embodiment at least two of R²⁰, R²¹, R²², or R²³ independently is an alkoxy. In yet another embodiment at least one of R²⁰, R²¹, R²², or R²³ is methoxy. In still another embodiment each of R²¹ and R²² is methoxy. In another embodiment each of R²⁰ and R²³ is —H. In an embodiment at least one of R²⁴, R²⁵, R²⁶, R²⁷, or R²⁸ is a halogen. In a second embodiment at least one of R²⁴, R²⁵, R²⁶, R²⁷, or R²⁸ is —Cl. In another embodiment R²⁵ is —Cl. In a further embodiment each of R²⁴, R²⁶, R²⁷, and R²⁸ is —H. In a preferred embodiment the anti-fungal small molecule has the following structure:

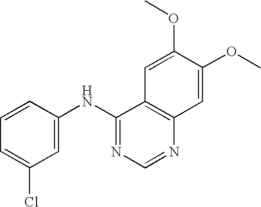

In an aspect of the invention an anti-fungal small molecule, analog or salt thereof has the following structure:

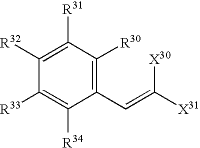

In an embodiment each of X³⁰ and X³¹ independently is —CN or a halogen; and each of R³⁰, R³¹, R³², R³³, and R³⁴ independently is one of —H, a halogen, an alkyl, —OH, or an alkoxy. In a second embodiment at least one of X³⁰ or X³¹ is —CN. In an embodiment each of X³⁰ and X³¹ is —CN. In an embodiment at least one of R³⁰, R³¹, R³², R³³, or R³⁴ is —OH. In a second embodiment R³² is —OH. In another embodiment at least one of R³⁰, R³¹, R³², R³³ or R³⁴ is an alkyl. In a further embodiment at least two of R³⁰, R³¹, R³², R³³, or R³⁴ independently is an alkyl. In yet another embodiment at least one of R³⁰, R³¹, R³², R³³, or R³⁴ is t-butyl. In still another embodiment each of R³¹ and R³³ is t-butyl. In another embodiment each of R³⁰ and R³⁴ is —H. In a preferred embodiment the anti-fungal small molecule has the following structure:

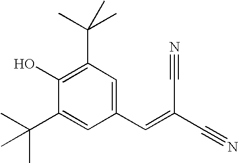

In an aspect of the invention an anti-fungal small molecule, analog or salt thereof has the following structure:

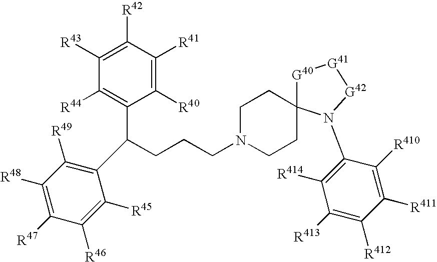

In an embodiment each of R⁴⁰, R⁴¹, R⁴², R⁴³, R⁴⁴, R⁴⁵, R⁴⁶, R⁴⁷, R⁴⁸, R⁴⁹, R⁴¹⁰, R⁴¹¹, R⁴¹², R⁴¹³, and R⁴¹⁴ independently is one of —H, a halogen, an alkyl, —OH, or an alkoxy; and each of G⁴⁰, G⁴¹, and G⁴² independently is one of 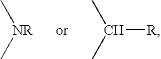
R at each occurrence independently being one of —H or an alkyl. In a second embodiment at least one of R⁴⁰, R⁴¹, R⁴², R⁴³, or R⁴⁴ is a halogen. In another embodiment at least one of R⁴⁰, R⁴¹, R⁴², R⁴³, or R⁴⁴ is —F. In a further embodiment R⁴² is —F. In yet another embodiment each of R⁴⁰, R⁴¹, R⁴³, and R⁴⁴ is —H. In still another embodiment at least one of R⁴⁵, R⁴⁶, R⁴⁷, R⁴⁸, or R⁴⁹ is a halogen. In a further embodiment at least one of R⁴⁵, R⁴⁶, R⁴⁷, R⁴⁸, or R⁴⁹ is —F. In another embodiment R⁴⁷ is —F. In yet another embodiment each of R⁴⁵, R⁴⁶, R⁴⁸, and R⁴⁹ is —H. In still another embodiment each of R⁴¹⁰, R⁴¹¹, R⁴¹², R⁴¹³, and R⁴¹⁴ is —H. In an embodiment at least one of G⁴⁰, G⁴¹, or G⁴² is 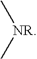
In a second embodiment G⁴¹ is 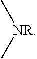
In another embodiment G⁴¹ is 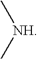
In a further embodiment G⁴⁰ is 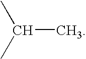
In yet another embodiment G⁴² is 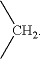
In a preferred embodiment the anti-fungal small molecule has the following structure:

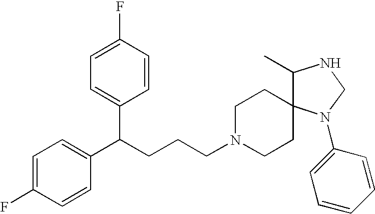

In an aspect of the invention an anti-fungal small molecule, analog or salt thereof has the following structure:

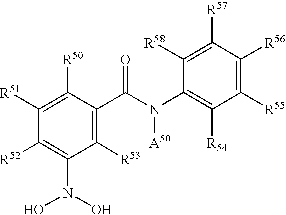

In an embodiment A⁵⁰ is one of —H or an alkyl; and each of R⁵⁰, R⁵¹, R⁵², R⁵³, R⁵⁴, R⁵⁵, R⁵⁶, R⁵⁷, and R⁵⁸ independently is one of is one of —H, a halogen, an alkyl, —OH, or an alkoxy. In a second embodiment at least one of R⁵⁰, R⁵¹, R⁵², or R⁵³ is a halogen. In another embodiment at least one of R⁵⁰, R⁵¹, R⁵², or R⁵³ is —Cl. In a further embodiment R⁵⁰ is —Cl. In yet another embodiment each of R⁵¹, R⁵², and R⁵³ is —H. In still a further embodiment each of R⁵⁴, R⁵⁵, R⁵⁶, R⁵⁷, and R⁵⁸ is —H. In another embodiment A⁵⁰ is —H. In a preferred embodiment the anti-fungal small molecule has the following structure:

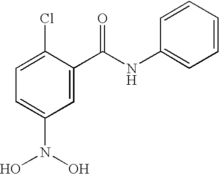

In an aspect of the invention an anti-fungal small molecule, analog or salt thereof has the following structure:

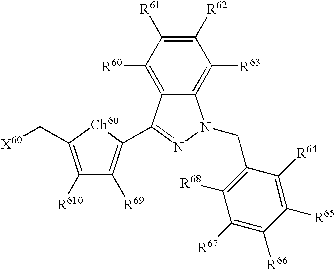

In an embodiment Ch⁶⁰ is a chalcogen; X⁶⁰ is one of —H, —OH, or a halogen; and each of R⁶⁰, R⁶¹, R⁶², R⁶³, R⁶⁴, R⁶⁵, R⁶⁶, R⁶⁷, R⁶⁸, R⁶⁹, and R⁶¹⁰ independently is one of —H, a halogen, an alkyl, —OH, or an alkoxy. In a second embodiment Ch⁶⁰ is oxygen. In another embodiment X⁶⁰ is —OH. In a further embodiment each of R⁶⁰, R⁶¹, R⁶², and R⁶³ is —H. In yet another embodiment each of R⁶⁴, R⁶⁵, R⁶⁶, R⁶⁷, and R⁶⁸ is —H. In still further embodiments each of R⁶⁹ and R⁶¹⁰ is —H. In a preferred embodiment the anti-fungal small molecule has the following structure:

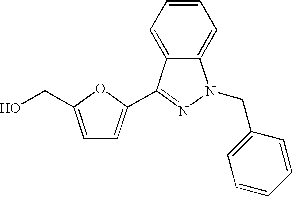

In an aspect of the invention an anti-fungal small molecule, analog or salt thereof has the following structure:

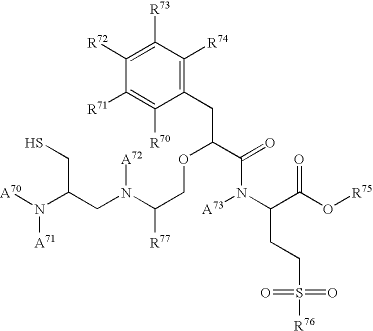

In an embodiment each of A⁷⁰, A⁷¹, A⁷², and A⁷³ independently is one of —H or an alkyl; and each of R⁷⁰, R⁷¹, R⁷², R⁷³, R⁷⁴, R⁷⁵, R⁷⁶, and R⁷⁷ independently is one of —H, a halogen, an alkyl, —OH, or an alkoxy. In a second embodiment each of A⁷⁰ and A⁷¹ is —H. In another embodiment A⁷² is —H. In a further embodiment A⁷³ is —H. In yet another embodiment each of R⁷⁰, R⁷¹, R⁷², R⁷³, and R⁷⁴ is —H. In still another embodiment R⁷⁵ is an alkyl. In another embodiment R⁷⁵ is isopropyl. In a further embodiment R⁷⁶ is an alkyl. In yet another embodiment R⁷⁶ is methyl. In still another embodiment R⁷⁷ is an alkyl. In still further embodiments R⁷⁷ is sec-butyl. In a preferred embodiment the anti-fungal small molecule has the following structure:

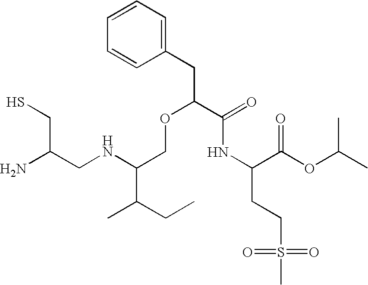

In an aspect of the invention an anti-fungal small molecule, analog or salt thereof has the following structure:

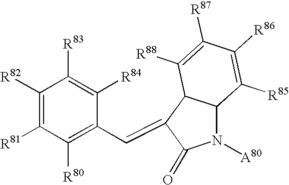

In an embodiment A⁸⁰ independently is one of —H or an alkyl; and each of R⁸⁰, R⁸¹, R⁸², R⁸³, R⁸⁴, R⁸⁵, R⁸⁶, R⁸⁷, and R⁸⁸ independently is one of —H, a halogen, an alkyl, —OH, or an alkoxy. In a second embodiment A⁸⁰ is —H. In another embodiment at least one of R⁸⁰, R⁸¹, R⁸², R⁸³, or R⁸⁴ is a halogen. In a further embodiment at least two of R⁸⁰, R⁸¹, R⁸², R⁸³, or R⁸⁴ independently is a halogen. In yet another embodiment at least one of R⁸⁰, R⁸¹, R⁸², R⁸³, or R⁸⁴ is —Br. In another embodiment each of R³¹ and R³³ is —Br. In still another embodiment at least one of R⁸⁰, R⁸¹, R⁸², R⁸³, or R⁸⁴ is —OH. In further embodiments R⁸² is —OH. In another embodiment each of R⁸⁰ and R⁸⁴ is —H. In a further embodiment at least one of R⁸⁵, R⁸⁶, R⁸⁷, or R⁸⁸ is a halogen. In still further embodiments at least one of R⁸⁵, R⁸⁶, R⁸⁷, or R⁸⁸ is —I. In a further embodiment each of R⁸⁵, R⁸⁶, and R⁸⁸ is —H. In a preferred embodiment the anti-fungal small molecule has the following structure:

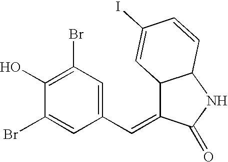

In an aspect of the invention an anti-fungal small molecule, analog or salt thereof has the following structure:

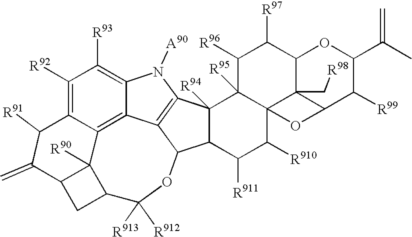

In an embodiment A⁹⁰ independently is one of —H or an alkyl; and each of R⁹¹, R⁹², R⁹³, R⁹⁴, R⁹⁵, R⁹⁶, R⁹⁷, R⁹⁸, R⁹⁹, R⁹¹⁰, R⁹¹¹, R⁹¹², and R⁹¹³ is independently is one of —H, a halogen, an alkyl, —OH, or an alkoxy. In a second embodiment A⁹⁰ is —H. In another embodiment R⁹⁰ is —OH. In a further embodiment at least one of R⁹² or R⁹³ is a halogen. In yet another embodiment at least one of R⁹² or R⁹³ is —Cl. In still further embodiments R⁹² is —Cl. In another embodiment R⁹³ is —H. In an embodiment at least one of R⁹⁴ or R⁹⁵ is an alkyl. In another embodiment each of R⁹⁴ and R⁹⁵ independently is an alkyl. In a further embodiment each of R⁹⁴ and R⁹⁵ is methyl. In yet another embodiment R⁹⁸ is —OH. In still another embodiment R⁹⁹ is —OH. In an embodiment at least one of R⁹¹² or R⁹¹³ is an alkyl. In another embodiment each of R⁹¹² and R⁹¹³ independently is an alkyl. In a further embodiment each of R⁹¹² and R⁹¹³ is methyl. In yet another embodiment R⁹¹ is —H. In still another embodiment each of R⁹⁶ and R⁹⁷ is —H. In a further embodiment each of R⁹¹⁰ and R⁹¹¹ is —H. In a preferred embodiment the anti-fungal small molecule has the following structure:

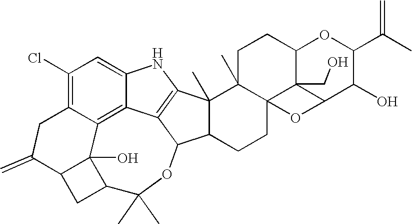

In an aspect of the invention an anti-fungal small molecule, analog or salt thereof has the following structure:

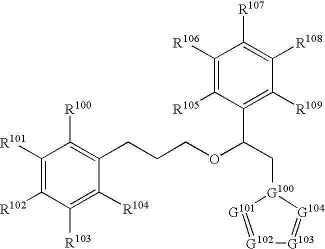

In one embodiment each of R¹⁰⁰, R¹⁰², R¹⁰³, R¹⁰⁴, R¹⁰⁵, R¹⁰⁶, and R¹⁰⁷, R¹⁰⁸, and R¹⁰⁹ independently is one of —H, a halogen, an alkyl, —OH, or an alkoxy; and each of G¹⁰⁰, G¹⁰¹, G¹⁰², G¹⁰³, and G¹⁰⁴ independently is one of N, NH⁺ or CR, R at each occurrence independently being one of —H or an alkyl. In a second embodiment only one of G¹⁰⁰, G¹⁰¹, G¹⁰², G¹⁰³, and G¹⁰⁴ is NH⁺. In another embodiment G¹⁰³ is NH⁺. In a further embodiment G¹⁰⁰ is N. In yet another embodiment each of G¹⁰¹, G¹⁰², and G¹⁰⁴ is CH. In still another embodiment at least one of R¹⁰⁰, R¹⁰², R¹⁰³, or R¹⁰⁴ is an alkoxy. In yet a further embodiment at least one of R¹⁰⁰, R¹⁰², R¹⁰³, or R¹⁰⁴ is methoxy. In still a further embodiment R¹⁰² is methoxy. In another embodiment at least one of R¹⁰⁵, R¹⁰⁶, R¹⁰⁷, R¹⁰⁸, or R¹⁰⁹ is an alkoxy. In still another embodiment at least one of R¹⁰⁵, R¹⁰⁶, R¹⁰⁷, R¹⁰⁸, or R¹⁰⁹ is methoxy. In a further embodiment R¹⁰⁷ is methoxy. In another embodiment the anti-fungal small molecule has the following structure: 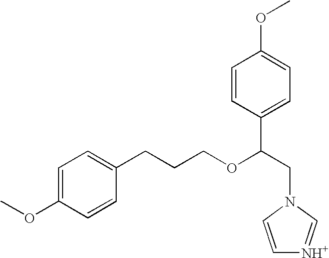
In a preferred embodiment the anti-fungal small molecule has the following structure:

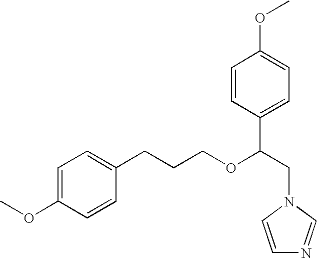

In an aspect of the invention an anti-fungal small molecule, analog or salt thereof has the following structure:

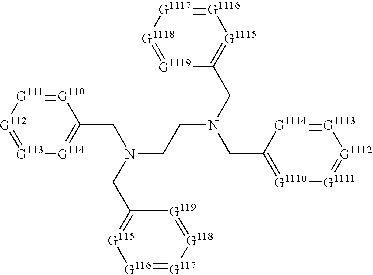

In an embodiment each of G¹¹⁰, G¹¹¹, G¹¹², G¹¹³, G¹¹⁴, G¹¹⁵, G¹¹⁶, G¹¹⁷, G¹¹⁸, G¹¹⁹, G¹¹¹⁰, G¹¹¹¹, G¹¹¹², G¹¹¹³, G¹¹¹⁴, G¹¹¹⁵, G¹¹¹⁶, G¹¹¹⁷, G¹¹¹⁸, and G¹¹¹⁹ independently is one of N or CR, R at each occurrence independently being one of —H or an alkyl. In a second embodiment at least one of G¹¹⁰, G¹¹¹, G¹¹², G¹¹³, or G¹¹⁴ is N. In another embodiment at least one of G¹¹⁵, G¹¹⁶, G¹¹⁷, G¹¹⁸, or G¹¹⁹ is N. In a further embodiment at least one of G¹¹¹⁰, G¹¹¹¹, G¹¹¹², G¹¹¹³, or G¹¹¹⁴ is N. In yet another embodiment at least one of G¹¹¹⁵, G¹¹¹⁶, G¹¹¹⁷, G¹¹¹⁸, or G¹¹¹⁹ is N. In still another embodiment G¹¹⁴ is N. In another embodiment G¹¹⁹ is N. In a further embodiment G¹¹¹⁴ is N. In yet another embodiment G¹¹¹⁹ is N. In an embodiment each of G¹¹⁰, G¹¹¹, G¹¹², G¹¹³, G¹¹⁵, G¹¹⁶, G¹¹⁷, G¹¹⁸, G¹¹¹⁰, G¹¹¹¹, G¹¹¹², G¹¹¹³, G¹¹¹⁵, G¹¹¹⁶, G¹¹¹⁷, and G¹¹¹⁸ is CH. In a preferred embodiment the anti-fungal small molecule has the following structure:

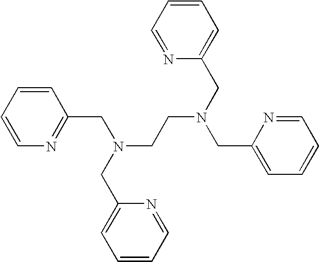

In an aspect of the invention an anti-fungal small molecule, analog or salt thereof has the following structure:

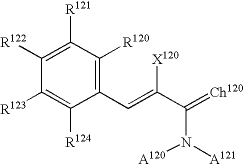

In an embodiment Ch¹²⁰ is a chalcogen; each of A¹²⁰ and A¹²¹ independently is one of —H or an alkyl; X¹²⁰ is —CN or a halogen; and each of R¹²⁰, R¹²¹, R¹²², R¹²³, and R¹²⁴ independently is one of —H, a halogen, an alkyl, —OH, or an alkoxy. In a second embodiment Ch¹²⁰ is sulfur. In another embodiment X¹²⁰ is —CN. In a further embodiment at least one of R¹²⁰, R¹²¹, R¹²², R¹²³, or R¹²⁴ is —OH. In yet another embodiment at least two of R¹²⁰, R¹²¹, R¹²², R¹²³, or R¹²⁴ independently is —OH. In still further embodiments each of R¹²² and R¹²³ is —OH. In another embodiment each of R¹²⁰, R¹²¹, and R¹²⁴ is —OH. In a further embodiment at least one of A¹²⁰ or A¹²¹ is —H. In yet another embodiment each of A¹²⁰ and A¹²¹ is —H. In a preferred embodiment the anti-fungal small molecule has the following structure:

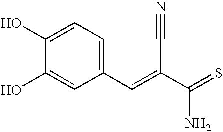

In an aspect of the invention an anti-fungal small molecule, analog or salt thereof has the following structure:

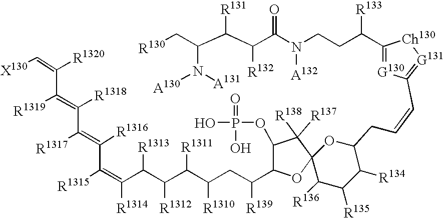

In an embodiment Ch¹³⁰ is a chalcogen; each of A¹³⁰, A¹³¹ and A¹³² independently is one of —H or an alkyl; X¹³⁰ is —CN or a halogen; each of R¹³⁰, R¹³¹, R¹³², R¹³³, R¹³⁴, R¹³⁵, R¹³⁶, R¹³⁷, R¹³⁸, R¹³⁹, R¹³¹⁰, R¹³¹¹, R¹³¹², R¹³¹³, R¹³¹⁴, R¹³¹⁵, R¹³¹⁶, R¹³¹⁷, R¹³¹⁸, R¹³¹⁹, and R¹³²⁰ independently is one of —H, a halogen, an alkyl, —OH, or an alkoxy; and each of G¹³⁰ and G¹³¹ independently is one of N or CR, R being one of —H or an alkyl. In a second embodiment X¹³⁰ is —CN. In another embodiment Ch¹³⁰ is oxygen. In a further embodiment at least one of G¹³⁰ or G¹³¹ is N. In yet another embodiment G¹³⁰ is N. In another embodiment G¹³¹ is CH. In an embodiment at least one of A¹³⁰ or A¹³¹ is an alkyl. In a second embodiment each of A¹³⁰ and A¹³¹ independently is an alkyl. In another embodiment each of A¹³⁰ and A¹³¹ is methyl. In a further embodiment A¹³² is —H. In an embodiment R¹³⁰ is an alkoxy. In another embodiment R¹³⁰ is methoxy. In a further embodiment at least one of R¹³¹ or R¹³² is —OH. In yet another embodiment each of R¹³¹ and R¹³² is —OH. In still another embodiment R¹³³ is an alkyl. In a further embodiment R¹³³ is methyl. In another embodiment at least one of R¹³⁴, R¹³⁵, or R¹³⁶ is an alkyl. In a further embodiment R¹³⁴ is an alkyl. In yet another embodiment R¹³⁴ is methyl. In still another embodiment at least one of R¹³⁴, R¹³⁵, or R¹³⁶ is —OH. In an embodiment R¹³⁵ is —OH. In another embodiment R¹³⁶ is —H. In a further embodiment at least one R¹³⁷ or R¹³⁸ is an alkyl. In another embodiment each of R¹³⁷ and R¹³⁸ independently is an alkyl. In an embodiment each of R¹³⁷ and R¹³⁸ is methyl. In another embodiment R¹³⁹ is an alkoxy. In a further embodiment R¹³⁹ is methoxy.

In an embodiment at least one of R¹³¹⁰, R¹³¹¹, R¹³¹², R¹³¹³, or R¹³¹⁴ is —OH. In a second embodiment at least two of R¹³¹⁰, R¹³¹¹, R¹³¹², R¹³¹³, or R¹³¹⁴ independently s —OH. In another embodiment each of R¹³¹⁰ and R¹³¹² is —OH. In a further embodiment at least one of R¹³¹⁰, R¹³¹¹, R¹³¹², R¹³¹³, or R¹³¹⁴ is an alkyl. In yet another embodiment at least two of R¹³¹⁰, R¹³¹¹, R¹³¹², R¹³¹³, or R¹³¹⁴ independently is an alkyl. In still another embodiment at least one of R¹³¹⁰, R¹³¹¹, R¹³¹², R¹³¹³, or R¹³¹⁴ is methyl. In another embodiment each of R¹³¹¹ and R¹³¹³ is methyl. In yet another embodiment R¹³¹⁴ is —H. In an embodiment at least one of R¹³¹⁵, R¹³¹⁶, R¹³¹⁷, R¹³¹⁸, R¹³¹⁹, or R¹³²⁰ is an alkyl. In a second embodiment at least two of R¹³¹⁵, R¹³¹⁶, R¹³¹⁷, R¹³¹⁸, R¹³¹⁹, or R¹³²⁰ independently is an alkyl. In another embodiment at least three of R¹³¹⁵, R¹³¹⁶, R¹³¹⁷, R¹³¹⁸, R¹³¹⁹, or R¹³²⁰ independently is an alkyl. In a further embodiment at least one of R¹³¹⁵, R¹³¹⁶, R¹³¹⁷, R¹³¹⁸, R¹³¹⁹, or R¹³²⁰ is methyl. In yet another embodiment each of R¹³¹⁵, R¹³¹⁶, and R¹³²⁰ is methyl. In still another embodiment each of R¹³¹⁷, R¹³¹⁸, and R¹³¹⁹ is —H. In a preferred embodiment the anti-fungal small molecule has the following structure:

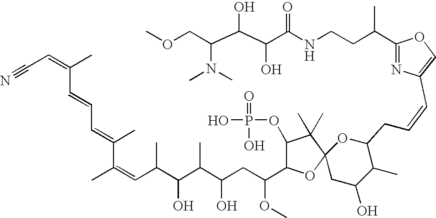

In an aspect of the invention an anti-fungal small molecule, analog or salt thereof has the following structure:

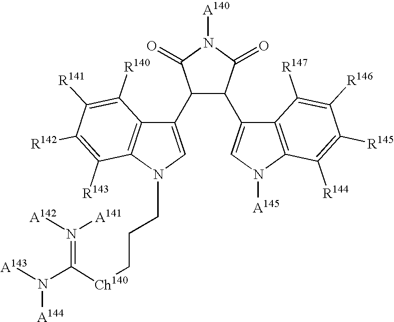

In an embodiment Ch¹⁴⁰ is a chalcogen; each of A¹⁴⁰, A¹⁴¹, A¹⁴², A¹⁴³, A¹⁴⁴, and A¹⁴⁵ independently is one of —H or an alkyl; and each of R¹⁴⁰, R¹⁴¹, R¹⁴², R¹⁴³, R¹⁴⁴, R¹⁴⁵, R¹⁴⁶, and R¹⁴⁷ independently is one of —H, a halogen, an alkyl, —OH, or an alkoxy. In a second embodiment Ch¹⁴⁰ is sulfur. In another embodiment A¹⁴⁰ is —H. In a further embodiment at least one of A¹⁴¹, A¹⁴², A¹⁴³, or A¹⁴⁴ is —H. In yet another embodiment at least two of A¹⁴¹, A¹⁴², A¹⁴³, or A¹⁴⁴ independently is —H. In still another embodiment each of A¹⁴¹, A ¹⁴², A¹⁴³, or A¹⁴⁴ is —H. In a further embodiment A¹⁴⁵ is an alkyl. In another embodiment A¹⁴⁵ is methyl. In still further embodiments each of R¹⁴⁰, R¹⁴¹, R¹⁴², and R¹⁴³ is —H. In yet another embodiment each of R¹⁴⁴, R¹⁴⁵, R¹⁴⁶, and R¹⁴⁷ is —H. In a further embodiment the anti-fungal small molecule has the following structure:

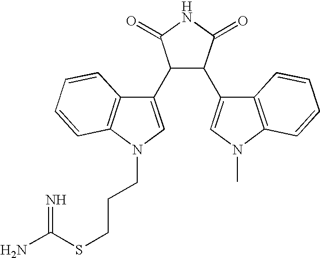

In a preferred embodiment the anti-fungal small molecule has the following structure:

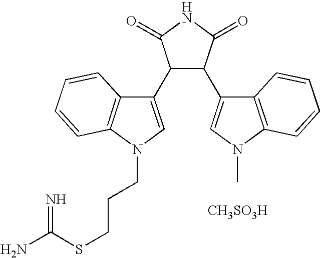

In an aspect of the invention an anti-fungal small molecule, analog or salt thereof has the following structure:

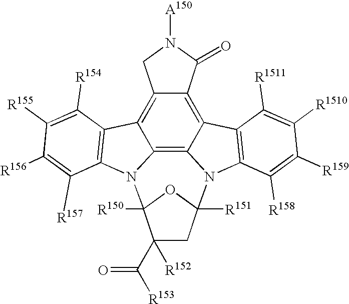

In an embodiment A¹⁵⁰ is one of —H or an alkyl; and each of R¹⁵⁰, R¹⁵¹, R¹⁵², R¹⁵³, R¹⁵⁴, R¹⁵⁵, R¹⁵⁶, R¹⁵⁷, R¹⁵⁸, R¹⁵⁹, R¹⁵¹⁰, and R¹⁵¹¹ independently is one of —H, a halogen, an alkyl, —OH, or an alkoxy. In a second embodiment A¹⁵⁰ is —H. In another embodiment at least one of R¹⁵⁰ or R¹⁵¹ is an alkyl. In a further embodiment R¹⁵⁰ is an alkyl. In yet another embodiment R¹⁵⁰ is methyl. In an embodiment R¹⁵¹ is —H. In another embodiment R¹⁵² is —OH. In a further embodiment R¹⁵³ is an alkoxy. In yet another embodiment R¹⁵³ is methoxy. In still further embodiments each of R¹⁵⁴, R¹⁵⁵, R¹⁵⁶, and R¹⁵⁷ is —H. In another embodiment each of R¹⁵⁸, R¹⁵⁹, R¹⁵¹⁰, and R¹⁵¹¹ is —H. In a preferred embodiment the anti-fungal small molecule has the following structure:

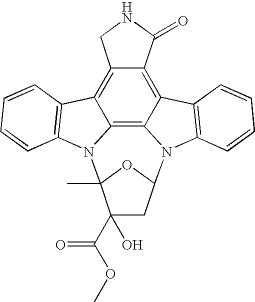

In an aspect of the invention an anti-fungal small molecule, analog or salt thereof has the following structure:

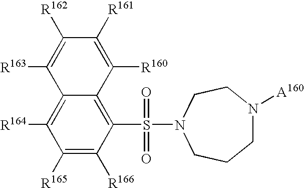

In an embodiment A¹⁶⁰ is one of —H or an alkyl; and each of R¹⁶⁰, R¹⁶¹, R¹⁶², R¹⁶³, R¹⁶⁴, R¹⁶⁵, and R¹⁶⁶ independently is one of —H, a halogen, an alkyl, —OH, or an alkoxy. In a second embodiment A¹⁶⁰ is —H. In another embodiment at least one of R¹⁶⁰, R¹⁶¹, R¹⁶², R¹⁶³, R¹⁶⁴, R¹⁶⁵, or R¹⁶⁶ is a halogen. In a further embodiment at least one of R¹⁶⁰, R¹⁶¹, R¹⁶², R¹⁶³, R¹⁶⁴, R¹⁶⁵, or R¹⁶⁶ is —Cl. In yet another embodiment R¹⁶³ is —Cl. In still another embodiment each of R¹⁶⁰, R¹⁶¹, R¹⁶², R¹⁶⁴, R¹⁶⁵, and R¹⁶⁶ is —H. In a preferred embodiment the anti-fungal small molecule has the following structure:

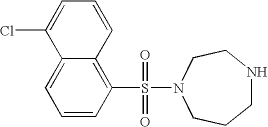

In another preferred embodiment the anti-fungal small molecule has the following structure:

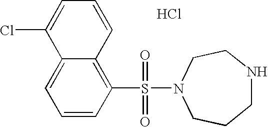

In an aspect of the invention an anti-fungal small molecule, analog or salt thereof has the following structure:

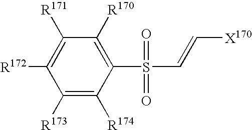

In an embodiment X¹⁷⁰ is —CN or a halogen; and each of R¹⁷⁰, R¹⁷¹, R¹⁷², R¹⁷³, and R¹⁷⁴ independently is one of —H, a halogen, an alkyl, —OH, or an alkoxy. In a second embodiment X¹⁷⁰ is —CN. In another embodiment at least one of R¹⁷⁰, R¹⁷¹, R¹⁷², R¹⁷³, or R¹⁷⁴ is an alkyl. In a further embodiment at least one of R¹⁷⁰, R¹⁷¹, R¹⁷², R¹⁷³, or R¹⁷⁴ is methyl. In yet another embodiment R¹⁷² is methyl. In a further embodiment each of R¹⁷⁰, R¹⁷¹, R¹⁷³, and R¹⁷⁴ is —H. In a preferred embodiment the anti-fungal small molecule has the following structure:

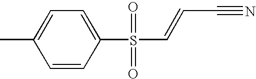

In an aspect of the invention an anti-fungal small molecule, analog or salt thereof has the following structure:

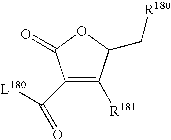

In an embodiment L¹⁸⁰ is an alkyl comprising at least 10 carbon atoms; and each of R¹⁸⁰ and R¹⁸¹ independently is one of —H, a halogen, an alkyl, —OH, or an alkoxy. In a second embodiment R¹⁸⁰ is —OH. In another embodiment R¹⁸¹ is —OH. In a further embodiment L¹⁸⁰ comprises at least 12 carbon atoms. In yet another embodiment L¹⁸⁰ comprises at least 15 carbon atoms. In an embodiment L¹⁸⁰ is a straight-chain alkyl. In another embodiment L¹⁸⁰ is a saturated alkyl. In a further embodiment L¹⁸⁰ is pentadecyl. In a preferred embodiment the anti-fungal small molecule has the following structure:

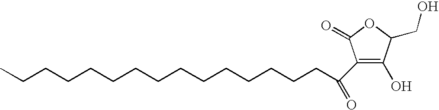

In an aspect of the invention an anti-fungal small molecule, analog or salt thereof has the following structure:

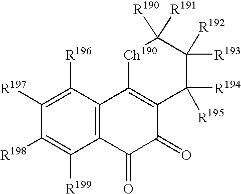

In an embodiment Ch¹⁹⁰ is a chalcogen; and each of R¹⁹⁰, R¹⁹¹, R¹⁹², R¹⁹³, R¹⁹⁴, R¹⁹⁵, R¹⁹⁶, R¹⁹⁷, R¹⁹⁸, and R¹⁹⁹ independently is one of —H, a halogen, an alkyl, —OH, or an alkoxy. In a second embodiment Ch¹⁹⁰ is oxygen. In another embodiment at least one of R¹⁹⁰, R¹⁹¹, R¹⁹², R¹⁹³, R¹⁹⁴, or R¹⁹⁵ is an alkyl. In a further embodiment at least two of R¹⁹⁰, R¹⁹¹, R¹⁹², R¹⁹³, R¹⁹⁴, or R¹⁹⁵ independently is an alkyl. In yet another embodiment at least one of R¹⁹⁰, R¹⁹¹, R¹⁹², R¹⁹³, R¹⁹⁴, or R¹⁹⁵ is methyl. In another embodiment each of R¹⁹⁰ and R¹⁹¹ is methyl. In a further embodiment each of R¹⁹², R¹⁹³, R¹⁹⁴, and R¹⁹⁵ is —H. In yet another embodiment each of R¹⁹⁶, R¹⁹⁷, R¹⁹⁸, and R¹⁹⁹ is —H. In a preferred embodiment the anti-fungal small molecule has the following structure:

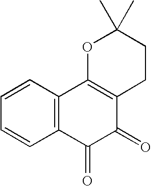

In an aspect of the invention an anti-fungal small molecule, analog or salt thereof has the following structure:

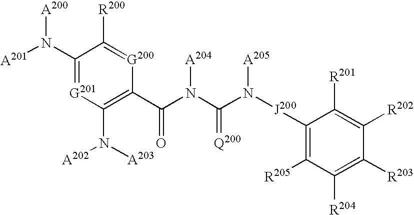

In an embodiment each of A²⁰⁰, A²⁰¹, A²⁰², A²⁰³, A²⁰⁴, and A²⁰⁵ independently is one of —H or an alkyl; each of R²⁰⁰, R²⁰¹, R²⁰², R²⁰³, R²⁰⁴, and R²⁰⁵ independently is one of —H, a halogen, an alkyl, —OH, or an alkoxy; each of G²⁰⁰ and G²⁰¹ independently is one of N or CR, R being one of —H or an alkyl; Q²⁰⁰ is one of ═N—R^{A} or 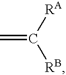
each of R^{A} and R^{B} independently being one of —H or an alkyl; and J²⁰⁰ is one of a covalent bond or an alkyl. In a second embodiment at least one of A²⁰⁰ or A²⁰¹ is —H. In another embodiment each of A²⁰⁰ and A²⁰¹ is —H. In a further embodiment at least one of A²⁰² or A²⁰³ is —H. In another embodiment each of A²⁰² or A²⁰³ is —H. In an embodiment A²⁰⁴ is —H. In another embodiment A²⁰⁵ is —H. In a further embodiment R²⁰⁰ is a halogen. In yet another embodiment R²⁰⁰ is —Cl. In still another embodiment Q²⁰⁰ is 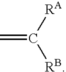
In a further embodiment Q²⁰⁰ is ═CH₂. In still further embodiments Q²⁰⁰ is ═N—R^{A}. In yet further embodiments Q²⁰⁰ is ═NH.

In an embodiment at least one of R²⁰¹, R²⁰², R²⁰³, R²⁰⁴, or R²⁰⁵ is a halogen. In a second embodiment at least two of R²⁰¹, R²⁰², R²⁰³, R²⁰⁴, or R²⁰⁵ independently is a halogen. In another embodiment at least one of R²⁰¹, R²⁰², R²⁰³, R²⁰⁴, or R²⁰⁵ is —Cl. In a further embodiment each of R²⁰¹ and R²⁰³ is —Cl. In yet another embodiment each of R²⁰¹, R²⁰⁴, and R²⁰⁵ is —H. In a further embodiment each of R²⁰¹, R²⁰², R²⁰³, R²⁰⁴, and R²⁰⁵ is —H. In still another embodiment at least one of G²⁰⁰ or G²⁰¹ is N. In yet another embodiment each of G²⁰⁰ and G²⁰¹ is N. In a further embodiment J²⁰⁰ is a covalent bond. In another embodiment J²⁰⁰ is an alkyl. In yet another embodiment J²⁰⁰ is —CH₂—. In a preferred embodiment the anti-fungal small molecule has the following structure:

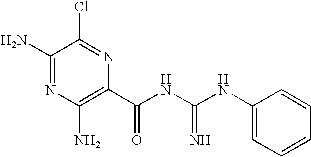

In aother preferred embodiment the anti-fungal small molecule has the following structure:

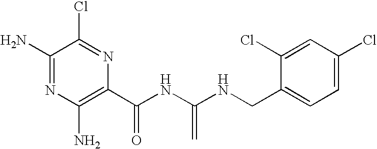

In an aspect of the invention an anti-fungal small molecule, analog or salt thereof has the following structure:

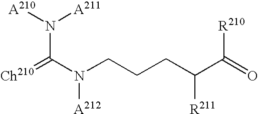

In an embodiment Ch²¹⁰ is a chalcogen; each of A²¹⁰, A²¹¹, and A²¹² independently is one of —H or an alkyl; and each of R²¹⁰ and R²¹¹ independently is one of —H, a halogen, an alkyl, —OH, or an alkoxy. In a second embodiment Ch²¹⁰ is sulfur. In another embodiment each of A²¹⁰ and A²¹¹ is —H. In a further embodiment A²¹² is —H. In yet another embodiment R²¹⁰ is OH. In still another embodiment R²¹¹ is an alkyl. In another embodiment R²¹¹ is methyl. In a preferred embodiment the anti-fungal small molecule has the following structure:

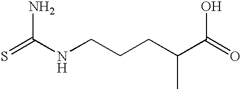

In an aspect of the invention an anti-fungal small molecule, analog or salt thereof has the following structure:

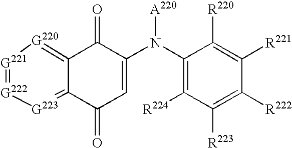

In an embodiment A²²⁰ is one of —H or an alkyl; each of R²²⁰, R²²¹, R²²², R²²³, and R²²⁴ independently is one of —H, a halogen, an alkyl, —OH, or an alkoxy; and each of G²²⁰, G²²¹, G²²², and G²²³ independently is one of N or CR, R at each occurrence independently being one of —H or an alkyl. In a second embodiment A²²⁰ is —H. In another embodiment each of R²²⁰, R²²¹, R²²², R²²³, and R²²⁴ is —H. In a further embodiment at least one of G²²⁰, G²²¹, G²²², or G²²³ is N. In yet another embodiment G²²³ is N. In still another embodiment each of G²²⁰, G²²¹, and G²²² is —H. In a preferred embodiment the anti-fungal small molecule has the following structure:

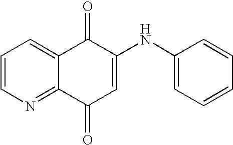

In an aspect of the invention an anti-fungal small molecule, analog or salt thereof has the following structure:

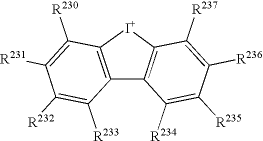

In an embodiment each of R²³⁰, R²³¹, R²³², R²³³, R²³⁴, R²³⁵, R²³⁶, and R²³⁷ independently is one of —H, a halogen, an alkyl, —OH, or an alkoxy. In a second embodiment each of R²³⁰, R²³¹, R²³², and R²³³ is —H. In another embodiment each of R²³⁴, R²³⁵, R²³⁶, and R²³⁷ is —H. In a preferred embodiment the anti-fungal small molecule has the following structure:

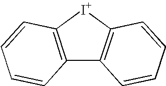

In an aspect of the invention an anti-fungal small molecule, analog or salt thereof has the following structure:

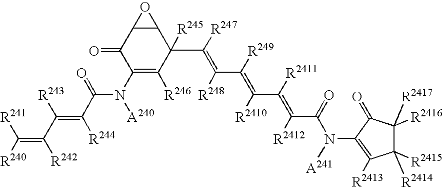

In an embodiment each of A²⁴⁰ and A²⁴¹ independently is one of —H or an alkyl; and each of R²⁴⁰, R²⁴¹, R²⁴², R²⁴³, R²⁴⁴, R²⁴⁵, R²⁴⁶, R²⁴⁷, R²⁴⁸, R²⁴⁹, R²⁴¹⁰, R²⁴¹¹, R²⁴¹², R²⁴¹³, R²⁴¹⁴, R²⁴¹⁵, R²⁴¹⁶, and R²⁴¹⁷ independently is one of —H, a halogen, an alkyl, —OH, or an alkoxy. In a second embodiment A²⁴⁰ is —H. In another embodiment A²⁴¹ is —H. In a further embodiment at least one of R²⁴⁰ or R²⁴¹ is an alkyl. In yet another embodiment R²⁴⁰ is 1-methylpenytl. In still another embodiment R²⁴¹ is —H. In another embodiment R²⁴² is an alkyl. In a further embodiment R²⁴² is methyl. In yet another embodiment R²⁴³ is —H. In still further embodiments R²⁴⁴ is an alkyl. In another embodiment R²⁴⁴ is methyl. In yet another embodiment R²⁴⁵ is —OH. In a further embodiment R²⁴⁶ is —H. In an embodiment each of R^{247, R}²⁴⁸, R²⁴⁹, R²⁴¹⁰, R²⁴¹¹, and R²⁴¹² is —H. In another embodiment at least one of R²⁴¹³, R²⁴¹⁴, R²⁴¹⁵, R²⁴¹⁶, or R²⁴¹⁷ is —OH. In a further embodiment R²⁴¹³ is —OH. In yet another embodiment each of R²⁴¹⁴, R²⁴¹⁵, R²⁴¹⁶, and R²⁴¹⁷ is —H. In a preferred embodiment the anti-fungal small molecule has the following structure:

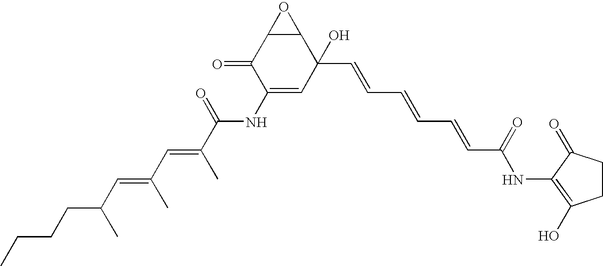

In an aspect of the invention an anti-fungal small molecule, analog or salt thereof has the following structure:

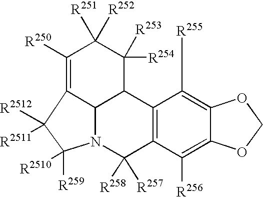

In an embodiment each of R²⁵⁰, R²⁵¹, R²⁵², R²⁵³, R²⁵⁴, R²⁵⁵, R²⁵⁶, R²⁵⁷, R²⁵⁸, R²⁵⁹, R²⁵¹⁰, R²⁵¹¹, and R²⁵¹² independently is one of —H, a halogen, an alkyl, —OH, or an alkoxy. In a second embodiment at least one of R²⁵⁰, R²⁵¹, R²⁵², R²⁵³, or R²⁵⁴ is —OH. In another embodiment at least two of R²⁵⁰, R²⁵¹, R²⁵², R²⁵³, or R²⁵⁴ independently is —OH. In a further embodiment R²⁵¹ is —OH. In yet another embodiment R²⁵³ is —OH. In still another embodiment each of R²⁵⁰, R²⁵², and R²⁵⁴ is —H. In another embodiment each of R²⁵⁵ and R²⁵⁶ is —H. In a further embodiment each of R²⁵⁷ and R²⁵⁸ is —H. In yet another embodiment each of R²⁵⁹, R²⁵¹⁰, R²⁵¹¹, and R²⁵¹² is —H. In a preferred embodiment the anti-fungal small molecule has the following structure:

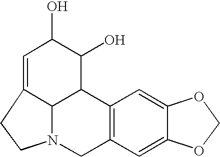

In an aspect of the invention an anti-fungal small molecule, analog or salt thereof has the following structure:

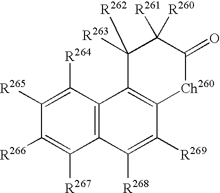

In an embodiment Ch²⁶⁰ is a chalcogen; and each of R²⁶⁰, R²⁶¹, R²⁶², R²⁶³, R²⁶⁴, R²⁶⁵, R²⁶⁶, R²⁶⁷, R²⁶⁸, and R²⁶⁹ independently is one of —H, a halogen, an alkyl, —OH, or an alkoxy. In a second embodiment Ch²⁶⁰ is oxygen. In another embodiment each of R²⁶⁰, R²⁶¹, R²⁶², and R²⁶³ is —H. In a further embodiment each of R²⁶⁴, R²⁶⁵, R²⁶⁶, and R²⁶⁷ is —H. In yet another embodiment each of R²⁶⁸ and R²⁶⁹ is —H. In a preferred embodiment the anti-fungal small molecule has the following structure:

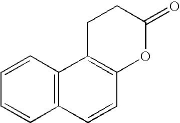

In an aspect of the invention an anti-fungal small molecule, analog or salt thereof has the following structure:

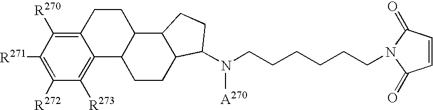

In an embodiment A²⁷⁰ is one of —H or an alkyl; and each of R²⁷⁰, R²⁷¹, R²⁷², and R²⁷³ independently is one of —H, a halogen, an alkyl, —OH, or an alkoxy. In a second embodiment A²⁷⁰ is —H. In another embodiment at least one of R²⁷⁰, R²⁷¹, R²⁷², or R²⁷³ is an alkoxy. In a further embodiment at least one of R²⁷⁰, R²⁷¹, R²⁷², or R²⁷³ is methoxy. In yet another embodiment R²⁷¹ is methoxy. In still another embodiment each of R²⁷⁰, R²⁷², and R²⁷³ is —H. In a preferred embodiment the anti-fungal small molecule has the following structure:

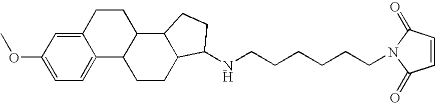

In an aspect of the invention an anti-fungal small molecule, analog or salt thereof has the following structure:

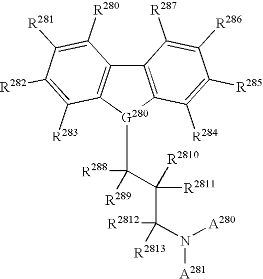

In an embodiment each of A²⁸⁰ and A²⁸¹ independently is one of —H or an alkyl; each of R²⁸⁰, R²⁸¹, R²⁸², R²⁸³, R²⁸⁴, R²⁸⁵, R²⁸⁶, R²⁸⁷, R²⁸⁸, R²⁸⁹, R²⁸¹⁰, R²⁸¹¹, R²⁸¹², and R²⁸¹³ independently is one of —H, a halogen, an alkyl, —OH, or an alkoxy; and G²⁸⁰ is one of N or CR, R being one of —H or an alkyl. In a second embodiment G²⁸⁰ is N. In another embodiment at least one of A²⁸⁰ or A²⁸¹ is an alkyl. In a further embodiment each of A²⁸⁰ and A²⁸¹ independently is an alkyl. In yet another embodiment at least one of A²⁸⁰ or A²⁸¹ is methyl. In an embodiment each of A²⁸⁰ and A²⁸¹ is methyl. In a further embodiment at least one of R²⁸⁰, R²⁸¹, R²⁸², or R²⁸³ is a halogen. In a second embodiment at least one of R²⁸⁰, R²⁸¹, R²⁸², or R²⁸³ is —Br. In another embodiment R²⁸¹ is —Br. In a further embodiment each of R²⁸⁰, R²⁸², and R²⁸³ is —H.

In an embodiment at least one of R²⁸⁴, R²⁸⁵, R²⁸⁶, or R²⁸⁷ is a halogen. In a second embodiment at least one of R²⁸⁴, R²⁸⁵, R²⁸⁶, or R²⁸⁷ is —Br. In another embodiment R²⁸⁶ is —Br. In a further embodiment each of R²⁸⁴, R²⁸⁵, and R²⁸⁷ is —H. In yet another embodiment R²⁸¹ and R²⁸⁶ are identical. In still another embodiment at least one of R²⁸⁸, R²⁸⁹, R²⁸¹⁰, R²⁸¹¹, R²⁸¹², or R²⁸¹³ is —OH. In another embodiment R²⁸¹⁰ is —OH. In a further embodiment each of R²⁸⁸, R²⁸⁹, R²⁸¹¹, R²⁸¹², and R²⁸¹³ is —H. In a preferred embodiment the anti-fungal small molecule has the following structure:

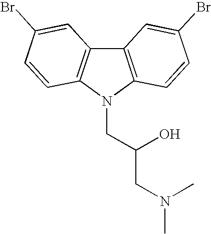

In an aspect of the invention an anti-fungal small molecule, analog or salt thereof has the following structure:

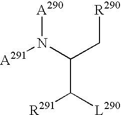

In an embodiment each of A²⁹⁰ and A²⁹¹ independently is one of —H or an alkyl; L²⁹⁰ is an alkyl comprising at least 10 carbon atoms; and each of R²⁹⁰ and R²⁹¹ independently is one of —H, a halogen, an alkyl, —OH, or an alkoxy. In a second embodiment at least one of A²⁹⁰ or A²⁹¹ is an alkyl. In another embodiment each of A²⁹⁰ and A²⁹¹ independently is an alkyl. In a further embodiment at least one of A²⁹⁰ or A²⁹¹ is methyl. In yet another embodiment each of A²⁹⁰ and A²⁹¹ is methyl. In an embodiment R²⁹⁰ is —OH. In another embodiment R²⁹¹ is —OH. In an embodiment L²⁹⁰ comprises at least 12 carbon atoms. In another embodiment L²⁹⁰ comprises at least 15 carbon atoms. In a further embodiment L²⁹⁰ is a straight-chain alkyl. In yet another embodiment L²⁹⁰ is an alkenyl. In still another embodiment L²⁹⁰ is 1-pentadecenyl. In a preferred embodiment the anti-fungal small molecule has the following structure:

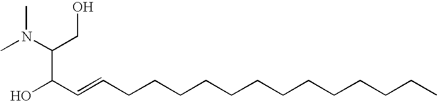

In an aspect of the invention an anti-fungal small molecule, analog or salt thereof has the following structure:

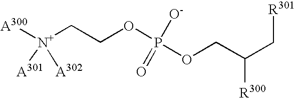

In an embodiment each of A³⁰⁰, A³⁰¹, and A³⁰² independently is one of —H or an alkyl; and each of R³⁰⁰ and R³⁰¹ independently is one of —H, a halogen, an alkyl, —OH, or an alkoxy. In a second embodiment at least one of A³⁰⁰, A³⁰¹, or A³⁰² is an alkyl. In another embodiment at least two of A³⁰⁰, A³⁰¹, or A³⁰² independently is an alkyl. In a further embodiment each of A³⁰⁰, A³⁰¹, and A³⁰² independently is an alkyl. In yet another embodiment at least one of A³⁰⁰, A³⁰¹, or A³⁰² is methyl. In still another embodiment each of A³⁰⁰, A³⁰¹, and A³⁰² is methyl. In an embodiment R³⁰⁰ is an alkoxy. In a second embodiment R³⁰⁰ is methoxy. In another embodiment R³⁰¹ is an alkoxy. In a further embodiment R³⁰¹ is an alkoxy comprising at least 10 carbon atoms. In yet another embodiment R³⁰¹ comprises at least 12 carbon atoms. In still another embodiment R³⁰¹ comprises at least 14 carbon atoms. In a further embodiment R³⁰¹ comprises at least 16 carbon atoms. In still further embodiments R³⁰¹ comprises at least 18 carbon atoms. In another embodiment R³⁰¹ is a straight-chain alkoxy. In a further embodiment R³⁰¹ is a saturated alkoxy. In yet another embodiment R³⁰¹ is hexadecoxy. In a further embodiment R³⁰¹ is octadecoxy. In a preferred embodiment the anti-fungal small molecule has the following structure:

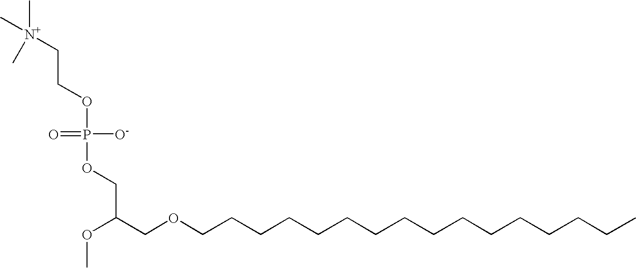

In another preferred embodiment the anti-fungal small molecule has the following structure:

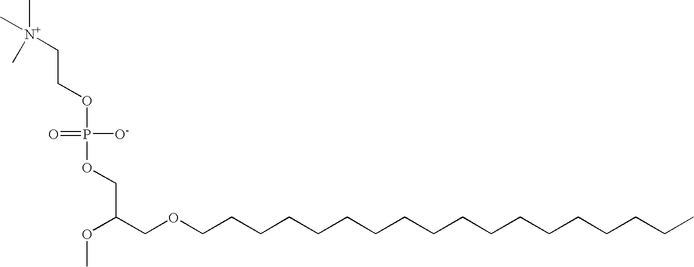

In an aspect of the invention an anti-fungal small molecule, analog or salt thereof has the following structure:

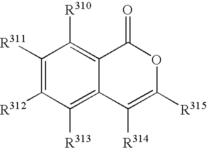

In an embodiment each of R³¹⁰, R³¹¹, R³¹², R³¹³, R³¹⁴, and R³¹⁵ independently is one of —H, a halogen, an alkyl, —OH, or an alkoxy. In a second embodiment at least one of R³¹⁰, R³¹¹, R³¹², R³¹³, R³¹⁴, or R³¹⁵ is a halogen. In another embodiment at least two of R³¹⁰, R³¹¹, R³¹², R³¹³, R³¹⁴, or R³¹⁵ independently is a halogen. In a further embodiment at least one of R³¹⁰, R³¹¹, R³¹², R³¹³, R³¹⁴, or R³¹⁵ is —Cl. In yet another embodiment each of R³¹⁴ and R³¹⁵ is —Cl. In still another embodiment each of R³¹⁰, R³¹¹, R³¹², and R³¹³ is —H. In a preferred embodiment the anti-fungal small molecule has the following structure:

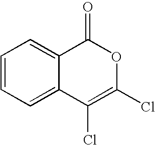

In an aspect of the invention an anti-fungal small molecule, analog or salt thereof is an alkanoic acid comprising at least 2 triple bonds. In an embodiment the alkanoic acid comprises at least 3 triple bonds. In a second embodiment the alkanoic acid comprises at least 4 triple bonds. In another embodiment the alkyl portion of the alkanoic acid is a straight-chain alkyl. In a further embodiment the alkyl comprises at least 6 carbon atoms. In yet another embodiment the alkyl comprises at least 8 carbon atoms. In still another embodiment the alkyl comprises at least 10 carbon atoms. In another embodiment the alkyl comprises at least 12 carbon atoms. In a further embodiment the alkyl comprises at least 14 carbon atoms. In yet another embodiment the alkyl comprises at least 16 carbon atoms. In still other embodiments the alkyl comprises at least 18 carbon atoms. In yet further embodiments the alkyl comprises at least 20 carbon atoms. In one preferred embodiment the anti-fungal small molecule has the following structure: 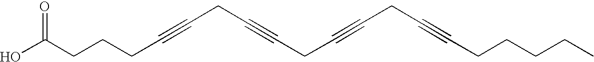
In another preferred embodiment the anti-fungal small molecule has the following structure:

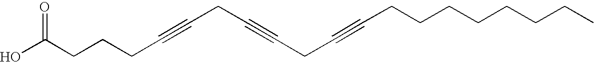

In an aspect of the invention an anti-fungal small molecule, analog or salt thereof has the following structure:

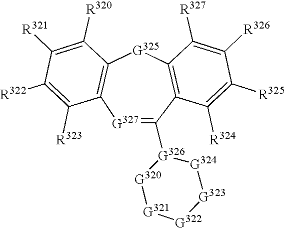

In an embodiment each of R³²⁰, R³²¹, R³²², R³²³, R³²⁴, R³²⁵, R³²⁶, and R³²⁷ independently is one of —H, a halogen, an alkyl, —OH, or an alkoxy; each of G³²⁰, G³²¹, G³²², G³²³, G³²⁴, and G³²⁵ independently is one of 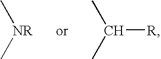
R at each occurrence independently being one of —H or an alkyl; and each of G³²⁶ and G³²⁷ is independently one of N or CR, R at each occurrence independently being one of —H or an alkyl. In a second embodiment at least one of R³²⁰, R³²¹, R³²², R³²³, R³²⁴, R³²⁵, R³²⁶, or R³²⁷ is a halogen. In another embodiment at least one of R³²⁰, R³²¹ , R³²², R³²³, R³²⁴, R³²⁵, R³²⁶, or R³²⁷ is —Cl. In a further embodiment R³²² is —Cl. In yet another embodiment each of R³²⁰, R³²¹, and R³²³ is —H. In still another embodiment each of R³²⁴, R³²⁵, R³²⁶, and R³²⁷ is —H. In another embodiment G³²⁷ is N. In a further embodiment G³²⁵ is NH. In yet another embodiment G³²⁶ is N. In an embodiment at least one of G³²⁰, G³²¹, G³²², G³²³ or G³²⁴ is 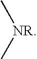
In another embodiment at least one of G³²⁰, G³²¹, G³²², G³²³, or G³²⁴ is 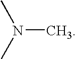
In a further embodiment G³²² is 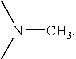
In yet another embodiment each of G³²⁰, G³²¹, G³²³, and G³²⁴ is 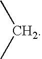
In a preferred embodiment the anti-fungal small molecule has the following structure:

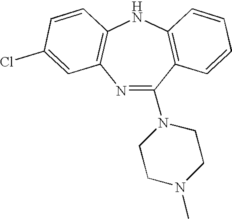

In an aspect of the invention an anti-fungal small molecule, analog or salt thereof has the following structure:

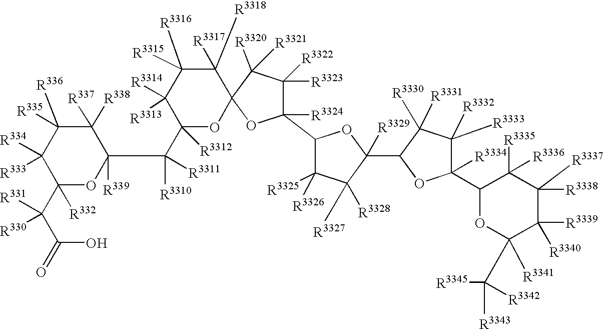

In an embodiment each of R³³⁰, R³³¹, R³³², R³³³, R³³⁴, R³³⁵, R³³⁶, R³³⁷, R³³⁸, R³³⁹, R³³¹⁰, R³³¹¹, R³³¹², R³³¹³, R³³¹⁴, R³³¹⁵, R³³¹⁶, R³³¹⁷, R³³¹⁸, R³³¹⁹, R³³²⁰, R³³²¹, R³³²², R³³²³, R³³²⁴, R³³²⁵, R³³²⁶, R³³²⁷, R³³²⁸, R³³²⁹, R³³³⁰, R³³³¹, R³³³², R³³³³, R³³³⁴, R³³³⁵, R³³³⁶, R³³³⁷, R³³³⁸, R³³³⁹, R³³⁴⁰, R³³⁴¹, and R³³⁴² independently is one of —H, a halogen, an alkyl, —OH, or an alkoxy. In a second embodiment R³³⁰ is an alkyl. In another embodiment R³³⁰ is methyl. In a further embodiment R³³¹ is —H. In yet another embodiment R³³² is —H. In an embodiment at least one of R³³³, R³³⁴, R³³⁵, R³³⁶, R³³⁷, or R³³⁸ is an alkyl. In another embodiment at least one of R³³³, R³³⁴, R³³⁵, R³³⁶, R³³⁷, or R³³⁸ is methyl. In a further embodiment R³³³ is methyl. In yet another embodiment each of R³³⁴, R³³⁵, R³³⁶, R³³⁷, and R³³⁸ is —H. In still another embodiment R³³⁹ is —H.

In an embodiment each of R³³¹⁰ and R³³¹¹ is —H. In a second embodiment R³³¹² is —H. In another embodiment at least one of R³³¹³, R³³¹⁴, R³³¹⁵, R³³¹⁶, R³³¹⁷, or R³³¹⁸ is an alkoxy. In a further embodiment at least one of R³³¹³, R³³¹⁴, R³³¹⁵, R³³¹⁶, R³³¹⁷, or R³³¹⁸ is methoxy. In yet another embodiment R³³¹⁵ is methoxy. In still another embodiment at least one of R³³¹³, R³³¹⁴, R³³¹⁵, R³³¹⁶, R³³¹⁷, or R³³¹⁸ is an alkyl. In an embodiment at least one of R³³¹³, R³³¹⁴, R³³¹⁵, R³³¹⁶, R³³¹⁷, or R³³¹⁸ is methyl. In another embodiment R³³¹⁷ is methyl. In a further embodiment each of R³³¹³, R³³¹⁴, R³³¹⁶, and R³³¹⁸ is —H. In yet another embodiment at least one of R³³²⁰, R³³²¹, R³³²², or R³³²³ is an alkyl. In still another embodiment at least one of R³³²⁰, R³³²¹, R³³²², or R³³²³ is methyl. In yet further embodiments R³³²⁰ is methyl.

In an embodiment each of R³³²¹, R³³²², and R³³²³ is —H. In a second embodiment R³³²⁴ is an alkyl. In another embodiment R³³²⁴ is methyl. In a further embodiment each of R³³²⁵, R³³²⁶, R³³²⁷, and R³³²⁸ is —H. In yet another embodiment R³³²⁹ is an alkyl. In still another embodiment R³³²⁹ is methyl. In an embodiment at least one of R³³³⁰, R³³³¹, R³³³², or R³³³³ is an alkyl. In another embodiment at least one of R³³³⁰, R³³³¹, R³³³², or R³³³³ is methyl. In a further embodiment R³³³⁰ is methyl. In yet another embodiment each of R³³³¹, R³³³², and R³³³³ is —H. In still another embodiment R³³³⁴ is —H. In an embodiment at least one of R³³³⁵, R³³³⁶, R³³³⁷, R³³³⁸, R³³³⁹, or R³³⁴⁰ is an alkyl. In another embodiment at least two of R³³³⁵, R³³³⁶, R³³³⁷, R³³³⁸, R³³³⁹, or R³³⁴⁰ independently is an alkyl. In a further embodiment at least one of R³³³⁵, R³³³⁶, R³³³⁷, R³³³⁸, R³³³⁹, or R³³⁴⁰ is methyl. In yet another embodiment each of R³³³⁵ and R³³³⁹ is methyl. In still another embodiment each of R³³³⁶, R³³³⁷, R³³³⁸, and R³³⁴⁰ is —H. In a further embodiment R³³⁴¹ is —OH. In another embodiment each of R³³⁴³ and R³³⁴⁴ is —H. In yet another embodiment R³³⁴⁵ is —OH. In a preferred embodiment the anti-fungal small molecule has the following structure:

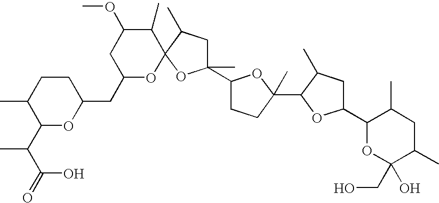

In an aspect of the invention an anti-fungal small molecule, analog or salt thereof has the following structure:

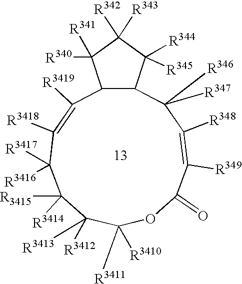

In an embodiment each of R³⁴⁰, R³⁴¹, R³⁴², R³⁴³, R³⁴⁴, R³⁴⁵, R³⁴⁶, R³⁴⁷, R³⁴⁸, R³⁴⁹, R³⁴¹⁰, R³⁴¹¹, R³⁴¹², R³⁴¹³, R³⁴¹⁴, R³⁴¹⁵, R³⁴¹⁶, R³⁴¹⁷, R³⁴¹⁸, and R³⁴¹⁹ independently is one of —H, a halogen, an alkyl, —OH, or an alkoxy. In a second embodiment at least one of R³⁴⁰, R³⁴¹, R³⁴², R³⁴³, R³⁴⁴, or R³⁴⁵ is —OH. In another embodiment R³⁴² is —OH. In a further embodiment each of R³⁴⁰, R³⁴¹, R³⁴³, R³⁴⁴, and R³⁴⁵ is —H. In yet another embodiment R³⁴⁶ is —OH. In still another embodiment R³⁴⁷ is —H. In an embodiment each of R³⁴⁸ and R³⁴⁹ is —H. In another embodiment at least one of R³⁴¹⁰, R³⁴¹¹, R³⁴¹², R³⁴¹³, R³⁴¹⁴, R³⁴¹⁵, R³⁴¹⁶, or R³⁴¹⁷ is an alkyl. In a further embodiment at least one of R³⁴¹⁰, R³⁴¹¹, R³⁴¹², R³⁴¹³, R³⁴¹⁴, R³⁴¹⁵, R³⁴¹⁶, or R³⁴¹⁷ is methyl. In yet another embodiment R³⁴¹⁰ is methyl. In still another embodiment each of R³⁴¹¹, R³⁴¹², R³⁴¹³, R³⁴¹⁴, R³⁴¹⁵, R³⁴¹⁶, and R³⁴¹⁷ is —H. In another embodiment each of R³⁴¹⁸ and R³⁴¹⁹ is —H. In a preferred embodiment the anti-fungal small molecule has the following structure:

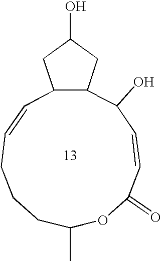

According to some aspects of the invention, the anti-fungal small molecules may be combined. In an embodiment of the invention the anti-fungal small molecule 4-(benzylidene-amino)-phenol may be combined with the anti-fungal small molecule 2-Chloro-5-nitro-N-phenylbenzamide analogs or salts thereof. In another embodiment of the invention the anti-fungal small molecule 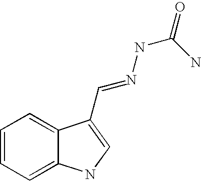
may be combined with the anti-fungal small molecule 8-Chloro-11-(4-methyl-1-piperazinyl)-5H-dibenzo[b,e][1,4]-diazepine analogs or salts thereof. In a further embodiment of the invention the anti-fungal small molecule 1-(2-isopropyl-5-methyl-cyclohexyloxy)-3-piperidin-1-yl-propan-2-ol may be combined with the anti-fungal small molecule 8-Chloro-11-(4-methyl-1-piperazinyl)-5H-dibenzo[b,e][1,4]-diazepine analogs or salts thereof. In a further embodiment the anti fungal small molecule 4-(benzylidene-amino)-phenol may be combined with the anti-fungal small molecule 8-Chloro-11-(4-methyl-1-piperazinyl)-5H-dibenzo[b,e][1,4]-diazepine analogs or salts thereof. In another embodiment of the invention the anti-fungal small molecule 1-(2-isopropyl-5-methyl-cyclohexyloxy)-3-piperidin-1-yl-propan-2-ol may be combined with the anti-fungal small molecule 2-Chloro-5-nitro-N-phenylbenzamide analogs or salts thereof. In an embodiment of the invention the anti-fungal small molecule 4-(benzylidene-amino)-phenol may be combined with the anti-fungal small molecule ethyl[2-amino-6-bromo-4-(1-cyano-2-ethoxy-2-oxoethyl)]-4H-chromene-3-carboxylate. It should be understood that any one or more of the anti-fungal small molecules of the invention may be combined with any other anti-fungal small molecule of the invention.

According to some aspects of the invention, the anti-fungal small molecules used in the methods for reducing the growth of a fungus or in the methods for treating fungal infection may exist in different isomeric forms. The anti-fungal small molecules may be used in the methods of the invention as a substantially isomerically-pure compound, or as a mixture of isomers. Preferably, isomerically-pure compounds are used. Isomerically-pure, as used herein, means that one isomer will be present in an amount ranging from 51 to 100%, preferably, more than 80%, more preferably, more than 90%, even more preferably, more than 95%, and even more preferably, more than 99% pure with respect to the other isomer or isomers present, but not with respect to other impurities or compounds that may be present. Isomer, as used herein, may refer to an E or Z isomer, and R or S isomer, an enantiomer, a diastereomer, or, in the case of anti-fungal small molecules with several diastereomers, a group of diastereomers, with respect to another group of diastereomers, which differ for example, with respect to just one stereocenter of the molecule.

As used herein, an “alkyl” is given its ordinary meaning as used in the field of organic chemistry. Alkyl or aliphatic groups typically contains any number of carbon atoms, for example, between 1 and 20 carbon atoms, between 1 and 15 carbon atoms, between 1 and 10 carbon atoms, or between 1 and 5 carbon atoms. In some embodiments, the alkyl group will contain at least 1 carbon atom, at least 2 carbon atoms, at least 3 carbon atoms, at least 4 carbon atoms, at least 5 carbon atoms, at least 6 carbon atoms, at least 7 carbon atoms, or at least 8 carbon atoms. Typically, an alkyl group is a non-cyclic structure. In certain embodiments, the alkyl group is a methyl group or an ethyl group.

The carbon atoms may be arranged in any configuration within the alkyl moiety, for example, as a straight chain (i.e., a n-alkyl such as methyl, ethyl, propyl, butyl, pentyl, hexyl, heptyl, octyl, nonyl, decyl, or undecyl) or a branched chain, for example, a t-butyl group, or an isoalkyl group such as isopropyl, isobutyl, ispentanyl, or isohexanyl. The alkyl moiety may contain none or any number of double or triple bonds within its structure, for example, as in an alkene, an alkyne, an alkadiene, an alkadiyne, an alkenyne, etc.

The alkyl group may contain any number of substituents. For example, the alkyl group may contain a halogen, an alkoxy (e.g., a methoxy, an ethoxy, a propoxy, an isopropoxy, a butoxy, a pentoxy, or the like), an amine (e.g., a primary, secondary, or tertiary amine, for example, an dimethylamine ethyl group), or a hydroxide as a substituent. As one example, if the alkyl group is a methyl group, then the methyl group may be substituted to form, for instance, a halogenated methyl group such as chloromethyl, bromomethyl, or iodomethyl. In some embodiments of the invention, more than one substituent may be present. For example, the alkyl group may have two or more halogen atoms (for example, two chlorine atoms, or a chlorine and a bromine atom), a halogen and an alkoxy group, or the like.

In some embodiments of the invention, the alkyl group may also contain one or more heteroatoms substituted within the alkyl group, such as a nitrogen atom (e.g., as in an amine such as a primary, secondary, or tertiary amine) or an oxygen atom (as in an ether moiety). However, in other embodiments of the invention, the main chain of the alkyl group is free of heteroatoms and includes carbon atoms. As used herein, the term “heteroatoms” refers to atoms that can replace carbon atoms within an alkyl group without affecting the connectivity of the alkyl group; these typically include oxygen and nitrogen atoms. Halogen atoms and hydrogen atoms are not considered to be heteroatoms; for example, a chlorine atom can replace a hydrogen atom within an alkyl group without affecting the connectivity of the alkyl group As used herein, a “non-heteroatom alkyl group” is an alkyl group which does not contain any atoms at the carbon positions other than carbon. Some structures are defined as being free of non-terminal heteroatoms. As used herein, a “non-terminal” atom is an atom within a structure that is connected to at least two different atoms having a valency greater than 1 (e.g., the atom is connected to two non-hydrogen and non-halogen atoms). For example, the oxygen in —CH₂—OH and the nitrogen atom in —CH₂—NH₂ are not connected to two different atoms having a valency greater than 1, and thus are not non-terminal heteroatoms.

The term “halogen,” or equivalently, “halogen atom,” is given its ordinary meaning as used in the field of chemistry. The halogens include fluorine, chlorine, bromine, iodine, and astatine. Preferably, the halogen atoms used in the present invention include one or more of fluorine, chlorine, bromine, or iodine. In certain embodiments of the invention, the halogen atoms found within the structure are fluorine, chlorine, and bromine; fluorine and chlorine; chlorine and bromine, or a single type of halogen atom.

In an aspect of the invention the anti-fungal small molecule is administered in an amount effective to reduce the growth of a fungus in a subject. As used herein an amount effective or effective amount, is an amount that is effective for producing some desired therapeutic effect in a subject at a reasonable benefit/risk ratio applicable to any medical treatment. An effective amount can mean an amount that reduces the symptoms in a subject or reduces detectable levels of fungus. Accordingly, in some embodiments, an effective amount prevents or minimizes disease symptoms or progression associated with fungal infection or fungal growth. An effective amount can also prevent, delay, or reduce the growth of a fungus or the appearance of symptoms associated with the fungal growth.

Actual dosage levels of the active components in the anti-fungal small molecules of the invention may be varied so as to obtain an amount of the active component that is effective to achieve the desired therapeutic response for a particular patient, anti-fungal small molecule, and mode of administration, without being toxic to the patient. The selected dosage level will depend upon a variety of factors including the activity of the particular anti-fungal small molecule of the present invention employed, the route of administration, the time of administration, the rate of excretion or metabolism of the particular agent being employed, the duration of the treatment, other drugs, agents and/or materials used in combination with the particular anti-fungal small molecule employed, the age, gender, weight, condition, general health and prior medical history of the patient being treated, and like factors well known in the medical arts.

A physician or veterinarian having ordinary skill in the art can readily determine and prescribe the effective amount of the anti-fungal small molecule required. For example, the physician or veterinarian could start doses of the anti-fungal small molecule(s) of the invention employed in the pharmaceutical composition at levels lower than that required to achieve the desired therapeutic effect and then gradually increase the dosage until the desired effect is achieved.

In an aspect of the invention the anti-fungal small molecule may be administered by any means suitable to obtain the desired therapeutic effect. In one aspect the desired effect is the reduction of growth of a fungus. The anti-fungal small molecule in this case is administered in a suitable manner to reduce the growth of a fungus. Administration routes include but are not limited to parenteral administration, topical, oral, nasal, aerosol and enema. Parenteral administration includes but is not limited to subcutaneous, intravenous, intramuscular, intraperitoneal, and intrasternal injection, or infusion techniques. Oral routes include but are not limited to oral, nasal, dermal, sublingual and inhalants. In one embodiment the anti-fungal small molecule is administered as a topical lotion or other formulation.

In an aspect of the invention the anti-fungal small molecule is administered over a suitable period of time in order to reduce the growth of the fungus. Generally, daily doses of an anti-fungal small molecule will be from about 0.01 milligrams/kg per day to 1000 milligrams/kg per day. It is expected that oral doses in the range of 0.5 to 50 milligrams/kg, or even 1-10 milligrams/kg per day, in one or several administrations per day, will yield the desired results. Dosage may be adjusted appropriately to achieve desired drug levels, local or systemic, depending upon the mode of administration. For example, it is expected that intravenous administration would be from an order to several orders of magnitude lower dose per day. It is expected that intravenous and other parenteral forms of administration will yield the desired results in the range of 0.1 to 10 milligrams/kg per day. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of anti-fungal small molecules. When in a topical form it may be applied once, twice or multiple times a day as required. A long-term sustained release implant may be particularly suitable for the treatment of chronic conditions. “Long-term” release, as used herein, means that the implant is constructed and arranged to deliver therapeutic levels of the active ingredient for at least 7 days, and preferably 30-60 days. The implant may be positioned at the site of infection. Long-term sustained release implants are well-known to those of ordinary skill in the art.

In another aspect, the present invention provides “pharmaceutically acceptable” compositions, that include a therapeutically effective amount of one or more of the anti-fungal small molecules described herein, formulated together with one or more pharmaceutically acceptable carriers (additives) and/or diluents. The pharmaceutical compositions of the present invention may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, drops, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, drops, gels, or a controlled-release patch or spray applied to the skin, lungs, or oral cavity; intravaginally or intrarectally, for example, as a pessary, cream or foam; sublingually; ocularly; transdermally; or nasally, pulmonary and to other mucosal surfaces.

In one aspect of the invention topical lotion formulations are provided. A topical lotion comprises an anti-fungal small molecule and a topical carrier. Topical carriers include but are not limited to creams, ointments, drops, gels, or a controlled-release patch or spray applied to the skin, lungs, or oral cavity. A topical carrier also includes a formulation administered intravaginally or intrarectally, for example, as a pessary, cream or foam, sublingually, ocularly, transdermally, or nasally, pulmonary, oralpharyngeal administration or to other mucosal surfaces. Suitable carrier components include, but are not limited to, mineral oil, sorbitan monostearate, polysorbate 60, cetyl esters wax, cetearyl alcohol, 2-octyldodecanol, benzyl alcohol, liquid petroleum, white petroleum, propylene glycol, polyoxyethylene polyoxypropylene compound, emulsifying wax and water.

The phrase “pharmaceutically acceptable” is employed herein to refer to those anti-fungal small molecules, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject extract from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; sterile distilled water; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active component which can be combined with a carrier material to produce a single dosage form will vary depending upon the host being treated, and the particular mode of administration. The amount of active component that can be combined with a carrier material to produce a single dosage form will generally be that amount of the extract which produces a therapeutic effect. Generally, this amount will range from about 1% to about 99% of active component, preferably from about 5% to about 80%, most preferably from about 40% to about 60%.

In certain embodiments, a formulation of the present invention comprises an excipient selected from the group consisting of cyclodextrins, liposomes, micelle forming agents, e.g., bile acids, and polymeric carriers, e.g., polyesters and polyanhydrides; and an anti-fungal small molecule of the present invention.

The anti-fungal small molecules of the invention may optionally be delivered with other antifungal agents in the form of antifungal cocktails, or individually, yet close enough in time to have an effect on the treatment of the infection. An antifungal agent may be delivered simultaneously, concurrently or sequentially to an anti-fungal small molecule. An antifungal cocktail is a mixture of any one of the above-described anti-fungal small molecules with another antifungal drug which may or may not be an anti-fungal small molecule of the invention. The use of such cocktails in pharmaceutical preparations is routine. In an embodiment, a common administration vehicle (e.g. lotion, gel, tablet, implants, injectable solution, injectable liposome solution, etc.) could contain both the anti-fungal small molecule of the invention and the other antifungal agent(s). The anti-fungal agent in combination with an anti-fungal small molecule is delivered in an amount effective to reduce the growth of a fungus in a subject or to produce some other desired therapeutic effect as described herein. The anti-fungal agent either alone or in combination with an anti-fungal small molecule, may or may not be in an amount effective.

Antifungal agents include but are not limited to Acrisorcin; Ambruticin; Amphotericin B; Azaconazole; Azaserine; Basifungin; Bifonazole; Biphenamine Hydrochloride; Bispyrithione Magsulfex; Butoconazole Nitrate; Calcium Undecylenate; Candicidin; Carbol-Fuchsin; Chlordantoin; Ciclopirox; Ciclopirox Olamine; Cilofungin; Cisconazole; Clotrimazole; Cuprimyxin; Denofungin; Dipyrithione; Doconazole; Econazole; Econazole Nitrate; Enilconazole; Ethonam Nitrate; Fenticonazole Nitrate; Filipin; Fluconazole; Flucytosine; Fungimycin; Griseofulvin; Hamycin; Isoconazole; Itraconazole; Kalafungin; Ketoconazole; Lomofungin; Lydimycin; Mepartricin; Miconazole; Miconazole Nitrate; Monensin; Monensin Sodium; Naftifine Hydrochloride; Neomycin Undecylenate; Nifuratel; Nifurmerone; Nitralamine Hydrochloride; Nystatin; Octanoic Acid; Orconazole Nitrate; Oxiconazole Nitrate; Oxifungin Hydrochloride; Parconazole Hydrochloride; Partricin; Potassium Iodide; Proclonol; Pyrithione Zinc; Pyrrolnitrin; Rapamycin; Rutamycin; Sanguinarium Chloride; Saperconazole; Scopafungin; Selenium Sulfide; Sinefungin; Sulconazole Nitrate; Tamoxifen; Terbinafine; Terconazole; Thiram; Ticlatone; Tioconazole; Tolciclate; Tolindate; Tolnaftate; Triacetin; Triafungin; Tunicamycin; Undecylenic Acid; Viridofulvin; Zinc Undecylenate; and Zinoconazole Hydrochloride.

### EXAMPLES

### Example 1

We provide numerous small molecules that inhibit either cell growth or the yeast-to-hyphal transition of the pathogenic yeast Candida albicans. These molecules have been identified from the ICCB/Harvard University Bioactive Knowns Collection. Analysis of the wide range of well-studied molecules in this collection has allowed us to systematically examine a variety of proteins and signaling pathways for their involvement in the budded-to-hyphal-form transition. For the screen, C. albicans cells were grown in YNB media that inhibits hyphal growth and then transferred to 384-well optical plates containing Spider media to induce the budded-to-hyphal transition and hyphal elongation. Next, small molecules were assayed for their ability to inhibit the Spider media-induced hyphal growth. Screening occurred on an inverted Nikon microscope with a computer driven XY stage with DIC/Hoffman optics, digital SPOT® camera and automatic shutter. Pictures were automatically taken of each well with the plate number and well position embedded in the picture through a script written for OpenLab® image analysis software from Improvision (INVIVO, from QED Imaging, Inc., Silver Spring, Md.).

Using this strategy, we screened 490 molecules from the ICCB collection that have known biological functions and/or cellular targets. The small molecules were added at time 0 and growth was allowed to continue for 4 h at 37° C. before the cells were fixed and observed microscopically. YNB media is well known in the art and contains yeast nitrogen base (DIFCO Labs., Detroit Mich.), glucose (US Biological, Swampscott, Mass.) and d-H₂O. Spider media is well known in the art and contains nutrient broth (DIFCO Labs., Detroit Mich.), mannitol (Sigma-Aldrich, St. Louis, Mo.), K₂HPO₄ (Sigma-Aldrich, St. Louis, Mo.) and d-H₂O.

Thirty nine of the screened molecules either inhibited C. albicans growth or inhibited the yeast-to-hyphal transition (Table 1). These molecules affected various signaling pathway components including those involved in Ca⁺² function, transmembrane receptors, protein kinases, and others. These data implicate these molecules as anti-fungal drugs and their associated signaling pathways in the regulation of the budded-to-hyphal-form transition. In addition, several molecules (YC-1, L-744,832) have displayed potent anti-tumor activity. These exciting results indicate that we can use our robust and reproducible screen to identify molecules with known biological functions that can inhibit growth or the yeast-to-hyphal transition. These molecules, some of which are already FDA approved for other medical uses, are potential therapeutic molecules against lethal C. albicans infections.

### Example 2

Cultures of Candida albicans were grown as described in Toenjes K. A. et al. (Antimicrobial Agents and Chemotherapy, 49(3):963-972, 2005). To induce hyphal growth, stationary-phase cultures were diluted into 5 ml of either YPD (1% yeast extract, 2% peptone, and 2% dextrose) plus 10% (vol/vol) fetal calf serum, Spider medium, Lee's medium (Lee, K. et al., Sabouraudia, 13:148-153, 1975), or M199 pH 8 medium (Sigma-Aldrich, St. Louis, Mo.) and grown at 37° C. with shaking at 250 rpm.

Quantification of inhibition of the budded-to-hyphal-form transition was accomplished by counting the numbers of individual budded cells versus the number of hyphae in the population. More than 100 cells were counted for each assay in duplicate and all assays were repeated four times. Individual hyphae were counted as one cell, although the hyphae usually consisted of multiple individual hyphal cells. The percentage of hyphae reported was normalized to the percentage of hyphae formed when no molecule was added.

For the synergy assay, two different molecules (100 μM) were added together and the assay was performed in 10% serum media to induce hyphal formation.

TABLE 2
Results:

| Molecule added | % hyphae in 10% serum |
|---|---|
| 235236 | 38% |
| GW9662 | 59% |
| 235236 + GW9662 | 0% |
| 235236 | 38% |
| Clozapine | 33% |
| 235236 + Clozapine | 4% |
| 105249 | 43% |
| HA14-1 | 34% |
| 105249 + HA14-1 | 0% |
| 121904 | 53% |
| Clozapine | 33% |
| 121904 + Clozapine | 11% |

Molecule 235236 showed a synergistic effect on hyphal growth in 10% serum with molecules GW9662 and Clozapine. Molecule 105249 showed a synergistic effect with HA14-1. Molecule 121904 showed a synergistic effect with Clozapine. Other combinations of molecules tested did not show synergistic effects on hyphal growth.

TABLE 1
Molecules that reduce fungal growth

| Molecule | Presumed Mode of Action |
|---|---|
| TMB-8 | Inhibits IP₃-induced intracellular Ca⁺² |
| | release |
| Nigericin | K⁺/H⁺ exchanger; inhibits intracellular |
| | Ca⁺² release |
| HA14-1 | Bcl-2 inhibitor; induces apoptosis and |
| | Ca⁺² release |
| Tyrophostin AG1478 | EGF receptor tyrosine kinase inhibitor |
| Tyrphostin 9 | PDGF receptor tyrosine kinase inhibitor |
| Clozapine | Dopamine receptor antagonist |
| Fluspiriline | Dopamine receptor antagonist |
| GW-9662 | Peroxisome proliferator-activated |
| | receptor (PPARγ) antagonist |
| YC-1 | NO-independent guanylyl cyclase |
| | activator; anti-tumor activity |
| L-744,832 | Peptidomimetic farnesyltransferase |
| | inhibitor |
| GW-5074 | Inhibitor of Raf-1 phosphorylation |
| 5,8,11,14-eicosatetraynoic acid | Prostaglandin and leukotriene antagonist |
| Penitrem A (tremortin A) | Large conductance Ca⁺² activated |
| | maxi-K channel blocker |
| SKF-96365 | Receptor mediated and voltage-gated |
| | Ca⁺² channel blocker |
| TPEN | Heavy metal chelator; low affinity for |
| | Ca⁺² and Mg⁺² |
| AG213 (Tyrphostin 47) | EGF receptor kinase inhibitor |
| Calyculin A | Protein phosphatase 1 and 2A inhibitor |
| Ro 31-8220 | Protein kinase inhibitor |
| K252A | Protein kinase inhibitor |
| ML-7 | Protein kinase inhibitor |
| ML-9 | Protein kinase inhibitor |
| BAY 11-7082 | Inhibits TNF-α-inducible phosphory- |
| | lation of lκB-α |
| RK-682 | Protein tyrosine phosphatase inhibitor |
| Beta-lapachone | DNA topoisomerase I inhibitor |
| Dichlorobenzamil | 8-Br-cGMP inhibitor |
| Thiocitrulline | Inhibitor of constitutive nitric oxide |
| | (NO) synthase |
| LY-83583 | Inhibits NO-induced activation of |
| | soluble guanylyl cylase C |
| Diphenyleneiodonium Cl | Endothelial nitric oxide synthase |
| | (eNOS) inhibitor |
| Manumycin A | Inhibits farnesyltransferase |
| Lycorine | Peptidyl transferase inhibitor |
| Brefeldin A | Inhibits protein translocation |
| Phenamil | Amiloride-sensitive sodium channel |
| | inhibitor |
| Splitomycin | Inhibitor of histone deacetylase activity |
| | of Sir2 protein |
| U73122 | Inhibits agonsit-induced phospholipase |
| | C activation |
| Wiskostatin | Inhibits actin filament assembly; |
| | inhibitor of N-WASP |
| N,N-dimethylsphingosine | Bioactive lipid |
| 1-hexyldecyl-2-methylglycero-3 | Bioactive lipid |
| phosphatidylcholine | |
| 3,4-dichloroisocoumarin | Inhibitor of multiple enzyme targets; |
| | factor D inhibitor |
| 5,8,11-eicosatriynoic acid | 5-LO, 12-LO, 15-LO and cyclo- |
| | oxygenase inhibitor |

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention. Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference in their entirety.

We claim:

1. A method for reducing growth of a fungus comprising contacting a fungal cell with an anti-fungal small molecule in an amount effective to reduce the growth of the fungal cell, wherein the anti-fungal small molecule is one or more of the following 8-(N,N-Diethylamino)-octyl-3,4,5-trimethoxybenzoate HCl, Ethyl[2-amino-6-bromo-4-(1-cyano-2-ethoxy-2-oxoethyl)]-4H-chromene-3-carboxylate, N-(3-Chlorophenyl)-6,7-dimethoxy-4-quinazolinamine, [[3,5-bis(1,1-Dimethylethyl)-4-hydroxyphenyl]methylene]propanedinitrile, 8-Chloro-11-(4-methyl-1-piperazinyl)-5H-dibenzo[b,e][1,4]-diazepine, 8-[4,4-bis(p-Fluorophenyl)butyl]-1-phenyl-1,3,8-triazino[4.5]decan-4-one, 2-Chloro-5-nitro-N-phenylbenzamide, 3-(5′-Hydroxymethyl-2′-furyl)-1-benzylindazole, (2S)-2-[[(2S)-2-[(2S,3S)-2-[(2R)-2-Amino-3-mercaptopropyl]amino]-3-methylpentyl]oxy]-1-oxo-3-phenylpropyl]amino]-4-(methylsulfonyl)-butanoic Acid 1-Methylethyl Ester, 3-(3,5-Dibromo-4-hydroxybenzyliden)-5-iodo-1,2-dihydroindol-2-one, 5,8,11,14-eicosatetrayonic acid, Penicillium palitans Penitrem A, 1-[β-[3-(4-Methoxyphenyl)propoxy]-4-methoxyphenethyl]-1H-imidazole HCl, N,N,N′,N′-tetrakis-(2-pyridylmethyl)-ethylenediamine, 3,4-Dihydroxy-a-cyanothiocinnamamide a-Cyano-3,4-dihydroxythiocinnamamide, Discodermia calyx Calyculin A, 3-[1-[3-(Amidinothio)propyl-1H-indol-3-yl]-3-(1-methyl-1H-indol-3-yl)maleimide Bisindolylmaleimide IX Methanesulfonate, Nocardiopsis K252A, 1-(5-Iodonapthalene-1-sulfonyl)homopiperazine, 1-(5-Chloronapthalene-1-sulfonyl)homopiperazine HCl, Streptomyces hygroscopicus Nigericin, γ,4-Dihydroxy-2-(6-hydroxy-1-heptenyl)-4-cyclopentanecrotonic acidγ-lactone, (E)3-[(4-Methylphenyl)sulfonyl]-2-propenenitrile, 3-Hexadecanoyl-5-hydroxymethyl-tetronic Acid, Tabebuia avellanedae Beta-lapachone, 2,4-Dichlorobenzamil, 2-Thioureido-L-norvaline, 6-Anilino-5,8-quinolinequinone, Diphenyleneiodonium Cl, Manumycin A, Lycorine, 3,5-Diamino-6-chloro-N-[imino(phenylamino)methyl]pyrazinecarboxamide, 1,2-Dihydro-3H-naphtho[2,1-b]pyran-3-one, 1-(6-(17β-3-methoxyestra-1,3,5(10)-trien-17-yl)amino)hexyl)-1H-pyrrole-2,5-dione, 1-(3,6-Dibromocarbazol-9-yl)-3-dimethylaminopropan-2-ol, N₁N-dimethylsphingosine, 1-hexadecyl-2-methylglycero-3-phosphatidylcholine, 1-octadecyl-2-methylglycero-3-phosphatidylcholine, 3,4dichloroisocoumarin, or 5,8,11-eicosatriyonic acid, 4-(benzylidene-amino)-phenol, or 1-(2-isopropyl-5-methyl-cyclohexyloxy)-3-piperidin-1-yl-propan-2-ol, or analogs or salts thereof.

2. The method of claim 2, wherein the method is a method for treating a subject having or at risk of having a fungal infection further comprising administering the anti-fungal small molecule to the subject.

3. The method of claim 2, wherein the fungal cell is selected from the group consisting of Candida albicans, Pneumocystis carinii, Saccharomyces cerevisiae, Aspergillus nidulans, Kluyveromyces lactis, Schizosaccharomyces pombe, Streptomyces lasaliensis, Streptomyces hygroscopicus, Candida tropicalis, Candida dubliniensis, Candida parapsilosis, Candida kefyr, Candida guilliermondii, Candida inconspicua, Candida famata, Candida glabrata, Candida krusei, Candida lusitaniae, Cryptococcus neoformans, Coccidioides immitis, and Hispolasma capsulatum.

4. The method of claim 2, wherein the fungal cell is a pathogenic yeast.

5. The method of claim 4, wherein the pathogenic yeast is Candida albicans.

6. The method of claim 2, wherein the anti-fungal small molecule is 8-(N,N-Diethylamino)-octyl-3,4,5-trimethoxybenzoate HCl, 8-(N,N-Diethylamino)-octyl-3,4,5-trimethoxybenzoate HCl analogs or salts thereof.

7. The method of claim 2, wherein the anti-fungal small molecule is Ethyl[2-amino-6-bromo-4-(1-cyano-2-ethoxy-2-oxoethyl)]-4H-chromene-3-carboxylate, Ethyl[2-amino-6-bromo-4-(1-cyano-2-ethoxy-2-oxoethyl)]-4H-chromene-3-carboxylate analogs or salts thereof.

8. The method of claim 2, wherein the anti-fungal small molecule is N-(3-Chlorophenyl)-6,7-dimethoxy-4-quinazolinamine, N-(3-Chlorophenyl)-6,7-dimethoxy-4-quinazolinamine analogs or salts thereof.

9. The method of claim 2, wherein the anti-fungal small molecule is [[3,5-bis(1,1-Dimethylethyl)-4-hydroxyphenyl]methylene]propanedinitrile, [[3,5-bis(1,1-Dimethylethyl)-4-hydroxyphenyl]methylene]propanedinitrile analogs or salts thereof.

10. The method of claim 2, wherein the anti-fungal small molecule is 8-Chloro-11-(4-methyl-1-piperazinyl)-5H-dibenzo[b,e][1,4]-diazepine, 8-Chloro-11-(4-methyl-1-piperazinyl)-5H-dibenzo[b,e][1,4]-diazepine analogs or salts thereof.

11. The method of claim 2, wherein the anti-fungal small molecule is 8-[4,4-bis(p-Fluorophenyl)butyl]-1-phenyl-1,3,8-triazino[4.5]decan-4-one, 8-[4,4-bis(p-Fluorophenyl)butyl]-1-phenyl-1,3,8-triazino[4.5]decan-4-one analogs or salts thereof.

12. The method of claim 2, wherein the anti-fungal small molecule is 2-Chloro-5-nitro-N-phenylbenzamide, 2-Chloro-5-nitro-N-phenylbenzamide analogs or salts thereof.

13. The method of claim 2, wherein the anti-fungal small molecule is 3-(5′-Hydroxymethyl-2′-furyl)-1-benzylindazole, 3-(5′-Hydroxymethyl-2′-furyl)-1-benzylindazole analogs or salts thereof.

14. The method of claim 2, wherein the anti-fungal small molecule is (2S)-2-[[(2S)-2-[(2S,3 S)-2-[(2R)-2-Amino-3-mercaptopropyl]amino]-3-methylpentyl]oxy]-1-oxo-3-phenylpropyl]amino]-4-(methylsulfonyl)-butanoic Acid 1-Methylethyl Ester, (2S)-2-[[(2S)-2-[(2S,3 S)-2-[(2R)-2-Amino-3-mercaptopropyl]amino]-3-methylpentyl]oxy]-1-oxo-3-phenylpropyl]amino]-4-(methylsulfonyl)-butanoic Acid 1-Methylethyl Ester analogs or salts thereof.

15. The method of claim 2, wherein the anti-fungal small molecule is 3-(3,5-Dibromo-4-hydroxybenzyliden)-5-iodo-1,2-dihydroindol-2-one, 3-(3,5-Dibromo-4-hydroxybenzyliden)-5-iodo-1,2-dihydroindol-2-one analogs or salts thereof.

16-18. (canceled)

19. The method of claim 2, wherein the anti-fungal small molecule is N,N,N′,N′-tetrakis-(2-pyridylmethyl)-ethylenediamine, N,N,N′,N′-tetrakis-(2-pyridylmethyl)-ethylenediamine analogs or salts thereof.

20. The method of claim 2, wherein the anti-fungal small molecule is 3,4-Dihydroxy-a-cyanothiocinnamamide a-Cyano-3,4-dihydroxythiocinnamamide, 3,4-Dihydroxy-a-cyanothiocinnamamide a-Cyano-3,4-dihydroxythiocinnamamide analogs or salts thereof.

21-32. (canceled)

33. The method of claim 2, wherein the anti-fungal small molecule is Manumycin A, Manumycin A analogs or salts thereof.

34-37. (canceled)

38. The method of claim 2, wherein the anti-fungal small molecule is 1-(3,6-Dibromocarbazol-9-yl)-3-dimethylaminopropan-2-ol, 1-(3,6-Dibromocarbazol-9-yl)-3-dimethylaminopropan-2-ol analogs or salts thereof.

39-43. (canceled)

44. The method of claim 2, wherein the anti-fungal small molecule is Streptomyces hygroscopicus Nigericin, Streptomyces hygroscopicus Nigericin analogs or salts thereof.

45. The method of claim 2, wherein the anti-fungal small molecule is γ,4-Dihydroxy-2-(6-hydroxy-1-heptenyl)-4-cyclopentanecrotonic acidγ-lactone, γ,4-Dihydroxy-2-(6-hydroxy-1-heptenyl)-4-cyclopentanecrotonic acidγ-lactone analogs or salts thereof.

46. The method of claim 2, wherein the fungal infection is a pathogenic yeast.

47. The method of claim 46, wherein the fungal infection is Candida albicans.

48. The method of claim 2, wherein the subject is a human.

49. The method of claim 2, wherein the subject is immunocompromised.

50. The method of claim 2, wherein the subject has had chemotherapy.

51. The method of claim 2, wherein the subject has AIDS.

52. The method of claim 2, wherein the subject has had a transplant.

53. The method of claim 2, wherein the subject has a central venous catheter.

54. The method of claim 2, wherein the anti-fungal small molecule is administered via injection, topical route, oral route, nasal route, aerosol, or enema route.

55. The method of claim 2, wherein the anti-fungal small molecule is administered via an oral route.

56. The method of claim 2, wherein the anti-fungal small molecule is administered via a topical route.

57. The method of claim 2, wherein the anti-fungal small molecule is 4-(benzylidene-amino)-phenol and 2-Chloro-5-nitro-N-phenylbenzamide analogs or salts thereof.

58. The method of claim 2, wherein the anti-fungal small molecule is 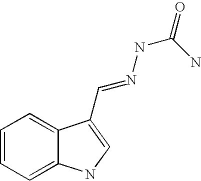
and 8-Chloro-11-(4-methyl-1-piperazinyl)-5H-dibenzo[b,e][1,4]-diazepine analogs or salts thereof.

59. The method of claim 2, wherein the anti-fungal small molecule is 1-(2-isopropyl-5-methyl-cyclohexyloxy)-3-piperidin-1-yl-propan-2-ol and 8-Chloro-11-(4-methyl-1-piperazinyl)-5H-dibenzo[b,e][1,4]-diazepine analogs or salts thereof.

60. The method of claim 2, wherein the anti-fungal small molecule is 4-(benzylidene-amino)-phenol and ethyl[2-amino-6-bromo-4-(1-cyano-2-ethoxy-2-oxoethyl)]-4H-chromene-3-carboxylate analogs or salts thereof.

61. A composition comprising
an anti-fungal small molecule and an anti-fungal agent, wherein the anti-fungal small molecule is one or more of the following 8-(N,N-Diethylamino)-octyl-3,4,5-trimethoxybenzoate HCl, Ethyl[2-amino-6-bromo-4-(1-cyano-2-ethoxy-2-oxoethyl)]-4H-chromene-3-carboxylate, N-(3-Chlorophenyl)-6,7-dimethoxy-4-quinazolinamine, [[3,5-bis(1,1-Dimethylethyl)-4-hydroxyphenyl]methylene]propanedinitrile, 8-Chloro-11-(4-methyl-1-piperazinyl)-5H-dibenzo[b,e][1,4]-diazepine, 8-[4,4-bis(p-Fluorophenyl)butyl]-1-phenyl-1,3,8-triazino[4.5]decan-4-one, 2-Chloro-5-nitro-N-phenylbenzamide, 3-(5′-Hydroxymethyl-2′-furyl)-1-benzylindazole, (2S)-2-[[(2S)-2-[(2S,3 S)-2-[(2R)-2-Amino-3-mercaptopropyl]amino]-3-methylpentyl]oxy]-1-oxo-3-phenylpropyl]amino]-4-(methylsulfonyl)-butanoic Acid 1-Methylethyl Ester, 3-(3,5-Dibromo-4-hydroxybenzyliden)-5-iodo-1,2-dihydroindol-2-one, 5,8,11,14-eicosatetrayonic acid, Penicillium palitans Penitrem A, 1-[β-[3-(4-Methoxyphenyl)propoxy]-4-methoxyphenethyl]-1H-imidazole HCl, N,N,N′,N′-tetrakis-(2-pyridylmethyl)-ethylenediamine, 3,4-Dihydroxy-a-cyanothiocinnamamide a-Cyano-3,4-dihydroxythiocinnamamide, Discodermia calyx Calyculin A, 3-[1-[3-(Amidinothio)propyl-1H-indol-3-yl]-3-(1-methyl-1H-indol-3-yl)maleimide Bisindolylmaleimide IX Methanesulfonate, Nocardiopsis K252A, 1-(5-Iodonapthalene-1-sulfonyl)homopiperazine, 1-(5-Chloronapthalene-1-sulfonyl)homopiperazine HCl, Streptomyces hygroscopicus Nigericin, γ,4-Dihydroxy-2-(6-hydroxy-1-heptenyl)-4-cyclopentanecrotonic acidγ-lactone, (E)3-[(4-Methylphenyl)sulfonyl]-2-propenenitrile, 3-Hexadecanoyl-5-hydroxymethyl-tetronic Acid, Tabebuia avellanedae Beta-lapachone, 2,4-Dichlorobenzamil, 2-Thioureido-L-norvaline, 6-Anilino-5,8-quinolinequinone, Diphenyleneiodonium Cl, Manumycin A, Lycorine, 3,5-Diamino-6-chloro-N-[imino(phenylamino)methyl]pyrazinecarboxamide, 1,2-Dihydro-3H-naphtho[2,1-b]pyran-3-one, 1-(6-(17β-3-methoxyestra-1,3,5(10)-trien-17-yl)amino)hexyl)-1H-pyrrole-2,5-dione, 1-(3,6-Dibromocarbazol-9-yl)-3-dimethylaminopropan-2-ol, N₁N-dimethylsphingosine, 1-hexadecyl-2-methylglycero-3-phosphatidylcholine, 1-octadecyl-2-methylglycero-3-phosphatidylcholine, 3,4-dichloroisocoumarin, or 5,8,11-eicosatriyonic acid, 4-(benzylidene-amino)-phenol, or 1-(2-isopropyl-5-methyl-cyclohexyloxy)-3-piperidin-1-yl-propan-2-ol, or analogs or salts thereof.

62-104. (canceled)

105. A topical lotion comprising an anti-fungal small molecule and a topical carrier, wherein the anti-fungal small molecule is one or more of the following 8-(N,N-Diethylamino)-octyl-3,4,5-trimethoxybenzoate HCl, Ethyl[2-amino-6-bromo-4-(1-cyano-2-ethoxy-2-oxoethyl)]-4H-chromene-3-carboxylate, N-(3-Chlorophenyl)-6,7-dimethoxy-4-quinazolinamine, [[3,5-bis(1,1-Dimethylethyl)-4-hydroxyphenyl]methylene]propanedinitrile, 8-Chloro-11-(4-methyl-1-piperazinyl)-5H-dibenzo[b,e][1,4]-diazepine, 8-[4,4-bis(p-Fluorophenyl)butyl]-1-phenyl-1,3,8-triazino[4.5]decan-4-one, 2-Chloro-5-nitro-N-phenylbenzamide, 3-(5′-Hydroxymethyl-2′-furyl)-1-benzylindazole, (2S)-2-[[(2S)-2-[(2S,3S)-2-[(2R)-2-Amino-3-mercaptopropyl]amino]-3-methylpentyl]oxy]-1-oxo-3-phenylpropyl]amino]-4-(methylsulfonyl)-butanoic Acid 1-Methylethyl Ester, 3-(3,5-Dibromo-4-hydroxybenzyliden)-5-iodo-1,2-dihydroindol-2-one, 5,8,11,14-eicosatetrayonic acid, Penicillium palitans Penitrem A, 1-[β-[3-(4-Methoxyphenyl)propoxy]-4-methoxyphenethyl]-1H-imidazole HCl, N,N,N′,N′-tetrakis-(2-pyridylmethyl)-ethylenediamine, 3,4-Dihydroxy-a-cyanothiocinnamamide a-Cyano-3,4-dihydroxythiocinnamamide, Discodermia calyx Calyculin A, 3-[1-[3-(Amidinothio)propyl-1H-indol-3-yl]-3-(1-methyl-1H-indol-3-yl)maleimide Bisindolylmaleimide IX Methanesulfonate, Nocardiopsis K252A, 1-(5-Iodonapthalene-1-sulfonyl)homopiperazine, 1-(5-Chloronapthalene-1-sulfonyl)homopiperazine HCl, Streptomyces hygroscopicus Nigericin, γ,4-Dihydroxy-2-(6-hydroxy-1-heptenyl)-4-cyclopentanecrotonic acidγ-lactone, (E)3-[(4-Methylphenyl)sulfonyl]-2-propenenitrile, 3-Hexadecanoyl-5-hydroxymethyl-tetronic Acid, Tabebuia avellanedae Beta-lapachone, 2,4-Dichlorobenzamil, 2-Thioureido-L-norvaline, 6-Anilino-5,8-quinolinequinone, Diphenyleneiodonium Cl, Manumycin A, Lycorine, 3,5-Diamino-6-chloro-N-[imino(phenylamino)methyl]pyrazinecarboxamide, 1,2-Dihydro-3H-naphtho[2,1-b]pyran-3-one, 1-(6-(17β-3-methoxyestra-1,3,5(10)-trien-17-yl)amino)hexyl)-1H-pyrrole-2,5-dione, 1-(3,6-Dibromocarbazol-9-yl)-3-dimethylaminopropan-2-ol, N₁N-dimethylsphingosine, 1-hexadecyl-2-methylglycero-3-phosphatidylcholine, 1-octadecyl-2-methylglycero-3-phosphatidylcholine, 3,4-dichloroisocoumarin, 5,8,11-eicosatriyonic acid, 4-(benzylidene-amino)-phenol, or 1-(2-isopropyl-5-methyl-cyclohexyloxy)-3-piperidin-1-yl-propan-2-ol, or analogs or salts thereof.

106-147. (canceled)
